SEQUENCE LISTING (12 sequences)

SEQ ID NO: 1 - protein, 211 aa, Trypanosoma cruzi
MGACGSKGSTSDKGLASDKDGKNAKDRKEAWEPIRQAIPREKTAEAKQRRIELFKKFDKNETGKLCYDEVYSGCLEVLRLDEFTSGVRDITKRAFDKARALGSKLENKGSEDFVEFLEFRLMLCYIYDFFELTVMFDEIDASGNMLVDEEEFKRAVPKLEAWGAKVEDPAALFKELDKNGTGSVTFDEFAAWASAVKLDADGDPDNVPESA

SEQ ID NO: 2 - DNA, 633 bp, Trypanosoma cruzi
atgggtgcttgtgggtcgaagggctcgacgagcgacaaggggttggcgagcgataaggacggcaagaacgccaaggaccgcaaggaagcgtgggagcccattcgccaggcgattcctcgtgagaagaccgccgaggcaaaacagcgccgcatcgagctcttcaagaagttcgacaagaacgagaccgggaagctgtgctacgatgaggtgtacagcggctgcctcgaggtgctgaggttggacgagttcacgtcgggagtgcgcgacatcacgaagcgtgcattcgacaaggcgagggccctgggcagcaagctggagaacaagggctccgaggactttgttgaatttctggagttccgtctgatgctgtgctacatctacgacttcttcgagctgacggtgatgttcgacgagattgacgcctccggcaacatgctggtcgacgaggaggagttcaagcgcgccgtgcccaagcttgaggcgtggggcgccaaggtcgaggatcccgcggcgctgttcaaggagctcgataagaacggcactgggtccgtgacgttcgacgagtttgctgcgtgggcttctgcagtcaaactggacgccgacggcgacccggacaacgtgccggagagcgcg

SEQ ID NO: 3 - protein, 604 aa, Mus musculus
MYPYDVPDYAVRWFHRDLSGPDAETLLKGRGVPGSFLARPSRKNQGDFSLSVRVDDQVTHIRIQNSGDFYDLYGGEKFATLTELVEYYTQQQGILQDRDGTIIHLKYPLNCSDPTSERWYHGHISGGQAESLLQAKGEPWTFLVRESLSQPGDFVLSVLNDQPKAGPGSPLRVTHIKVMCEGGRYTVGGSETFDSLTDLVEHFKKTGIEEASGAFVYLRQPYYATRVNAADIENRVLELNKKQESEDTAKAGFWEEFESLQKQEVKNLHQRLEGQRPENKSKNRYKNILPFDHSRVILQGRDSNIPGSDYINANYVKNQLLGPDENSKTYIASQGCLDATVNDFWQMAWQENTRVIVMTTREVEKGRNKCVPYWPEVGTQRVYGLYSVTNSREHDTAEYKLRTLQISPLDNGDLVREIWHYQYLSWPDHGVPSEPGGVLSFLDQINQRQESLPHAGPIIVHSSAGIGRTGTIIVIDMLMESISTKGLDCDIDIQKTIQMVRAQRSGMVQTEAQYKFIYVAIAQFIETTKKKLEIIQSQKGQESEYGNITYPPAVRSAHAKASRTSSKHKEEVYENVHSKSKKEEKVKKQRSADKEKNKGSLKRK

SEQ ID NO: 4 - DNA, 1812 bp, Mus musculus
atgtacccatatgacgttccagactacgcggtgaggtggtttcaccgggacctcagcgggcctgatgcagagaccctgctgaagggccggggagtccctgggagcttcctggctcggcccagccgcaagaaccagggtgacttctccctctcagtcagggtggatgatcaggtgactcatattcggatccagaactcaggggacttctatgacctgtacggaggggagaagtttgcgacgctgacagagctggtcgagtattacacgcagcagcagggcatcctgcaggaccgagatggcaccatcatccaccttaagtacccactgaactgctcggaccccaccagtgagaggtggtaccacggccacatatctggagggcaggcggagtcactgctgcaggccaagggcgagccctggacatttcttgtgcgtgagagtctcagccaacctggtgattttgtgctctctgtgctcaatgaccagcccaaggctggcccaggttccccgctcagggtcactcatatcaaggttatgtgtgagggtggacgctatactgtgggtggctcagagacgtttgacagcctcacagacctggtggagcacttcaagaagacagggattgaggaggcctcgggtgcctttgtctacctgcggcagccttactacgctactcgggtaaacgcagctgacattgagaatcgggtcttggaactgaacaagaagcaggagtcggaggacacagccaaggctggcttctgggaggagtttgagagtctacaaaagcaggaggtaaagaatctacaccaacgtctggaagggcagcggccagagaacaagagcaagaaccgctacaagaacattcttccctttgaccacagccgagtgatcctgcagggacgtgacagtaacatcccaggctctgactacatcaatgccaactacgtgaagaaccagctgctaggtccagatgagaactctaagacctacatcgccagccagggctgtctggatgccacagtcaatgacttctggcagatggcttggcaggagaacactcgtgtcatcgtcatgactaccagagaggtggagaaaggccggaacaaatgtgtcccatactggcccgaggtgggcactcagcgtgtctatggtctctactctgtgaccaacagtagggagcatgacacagcagaatacaaactgcgaacattacagatctccccactagacaatggggacctggttcgggagatatggcactaccagtacctgagctggcctgaccatggggttcccagtgagcctgggggtgtcctcagctttctggatcagatcaaccagcgacaggaaagtttgcctcatgcagggcccatcattgtgcattccagcgctggcatcggccgcacgggcaccatcatcgtcattgatatgcttatggaaagcatctccaccaaggggctagactgtgacattgatatccagaagaccatccagatggtacgagcacagcgctccggcatggtgcagaccgaggcccagtacaagtttatttacgtggccattgcccagttcatcgaaacgaccaagaagaaactggagatcatacaatcccagaagggccaggagtcggagtatgggaatatcacgtaccctcccgctgtgaggagtgcccacgccaaagcctcgcgtacttcctccaagcacaaggaggaggtgtacgaaaacgtgcatagcaagagcaagaaggaagagaaagtaaagaagcagcggtcggcagacaaggagaagaacaaaggttctctcaagaggaag

SEQ ID NO: 5 - protein, 589 aa, Trypanosoma cruzi
MERNSGDLQLPQEIAMLVPNKTQVVPKSGGEGKVKDIFASPALVRAGGVMIAFVEGRTKNKLFPEVIDLSSSDIVAGYIKAPETWQSLVAEVTKEYWQAHTVLESANNSNHRVGVARLPTGITRGNKVFLLVGSYEERREIDDYIWKAEAWNIKVIEGEATQSTEVQPTQPINWSEPKPLFQTDSPNNKGDLKEFLGGGGSGIVMGNGTLVFPLTAKDESNKVFSLITYSTDDGQKWEIPGGVSSVACRSPRVTEWEEGTLLMVTYCEDGRKVFESRDMGKTWTEAFGTLPGVWLKSGPELPEVSLRVDALITATIEGRKVMLYTQKVRHFLEVDEPNALHLWVTDNNRTFHLGPFSVDCAENKTFANTLLYSDDALHLLQAKGDHESTAVSLARLTEELNTINSVLSTWVQLDASFSESSIPTAGLVGFLSNTTSSGDTWIDGYRCMNATVTKAAKVENGFKFTGPGSRATWPVNSRWDIKQYGFVDYNFTIVAMATIHQVPSESTPLLGASLRGNKRTKLIGLSYGAGGKWETVYDGTKTVQGGTWEPGREYQVALMLQDGNKGFVYVDGVLVGNPAMLPTPEERWT

SEQ ID NO: 6 - DNA, 1767 bp, Trypanosoma cruzi
atggagagaaactcaggggatttgcaactgccgcaggagatcgccatgcttgtgccgaataagacgcaggtggtaccaaaaagtggtggtgaaggcaaagtgaaggatatctttgcttcacccgctctcgtccgtgctggtggagtgatgattgcatttgtcgaaggccgcaccaaaaataaactttttccagaagtgattgatctcagttcttctgatattgttgccggttacatcaaggctccagagacatggcagtctcttgttgctgaggtaaccaaagagtactggcaggcacacactgtccttgagagtgcgaataatagtaatcatcgtgtgggtgttgcgaggctacccaccggaattacaaggggcaataaagtgtttctgctagtggggagctatgaagagaggcgtgaaattgatgattatatttggaaggccgaggcatggaacattaaagtgattgagggtgaggccacgcagtccacggaagttcagccgactcaaccgatcaactggagtgaacccaaaccgctgttccaaactgactctcctaataataaaggtgacctaaaggaatttttgggtggtggtggctcaggaattgtgatggggaatggcacacttgtgtttcccctgacggcaaaggatgaaagtaataaagttttctccctaatcacttattcgacggacgacggccaaaagtgggagataccagggggcgtttcttctgtggcatgccgttccccccgcgtcaccgaatgggaggagggaacacttctcatggttacttattgcgaggatggccgcaaggtgtttgagtcgcgtgacatggggaaaacgtggacggaggcttttgggacactcccaggcgtgtggctcaaatcaggtccagaacttccggaagtaagtttgcgtgttgatgctctcatcaccgcgaccattgagggaaggaaggtcatgctgtacactcagaaagtgaggcattttctggaagtggatgaacccaacgcactccacctttgggtcacggacaacaaccgcacttttcatcttggaccgttttctgtggactgtgctgagaataagacgtttgccaacaccttgctgtactcggatgatgcgttgcaccttttacaagcgaagggcgatcatgaaagcacagccgtttcacttgcccgcctaacggaggagctgaatacgatcaattctgtccttagtacttgggtacagttggacgcttccttctccgagtcgtctatacccacggctggtctggttggattcctgtccaatacgacgtccagtggagacacgtggatcgacgggtaccgttgcatgaatgcaacggtgacgaaggcagcgaaggttgaaaatggtttcaagttcacggggcctgggtccagggcaacatggcccgtaaacagtcggtgggatattaaacagtacggctttgtggattacaacttcactattgtggcgatggcgactatacaccaggttccgagtgagagcactcctctgctgggtgcgagtctgaggggcaataagaggacgaagttaattggtttgtcgtacggtgcgggcggtaagtgggagacagtgtatgacgggacaaaaacagtacagggtggcacttgggagccggggagagaataccaggtggcgctcatgctgcaggacggcaacaagggcttcgtgtacgtggatggtgtgcttgtggggaacccggcgatgttaccaacacctgaggagcggtggact

SEQ ID NO: 7 - DNA, 20 bp, Artificial sequence
  Synthetic oligonucleotide
aatcggctgatcgttttcga

SEQ ID NO: 8 - DNA, 21 bp, Artificial sequence
  Synthetic oligonucleotide
aattcctccaagcagcggata

SEQ ID NO: 9 - DNA, 17 bp, Artificial sequence
  Synthetic oligonucleotide
cacacactggacaccaa

SEQ ID NO: 10 - DNA, 21 bp, Artificial sequence
  Synthetic oligonucleotide
caatgtgtccgtcgtggatct

SEQ ID NO: 11 - DNA, 22 bp, Artificial sequence
  Synthetic oligonucleotide
gtcctcagtgtagcccaagatg

SEQ ID NO: 12 - DNA, 23 bp, Artificial sequence
  Synthetic oligonucleotide
cgtgccgcctggagaaacctgcc

(19) United States
(12) Patent Application Publication
(10) Pub. No.: US 2021/0268080 A1
(43) Pub. Date: Sep. 2, 2021

(54) IMMUNE STIMULATING COMPOSITIONS FOR THE TREATMENT AND REVENTION OF INFECTIONS WITH INTRACELLULAR PATHOGENS

(71) Applicant: BAYLOR COLLEGE OF MEDICINE, Houston, TX (US)

(72) Inventors: William K. DECKER, Houston, TX (US); Vanaja KONDURI, Houston, TX (US); Kathryn M. JONES, Houston, TX (US); Jonathan M. LEVITT, Houston, TX (US); Peter J. HOTEZ, Houston, TX (US); Maria E. BOTTAZZI, Houston, TX (US)

(21) Appl. No.: 17/257,343

(22) PCT Filed: Jul. 3, 2019
(86) PCT No.: PCT/US2019/040467

§ 371 Date: Dec. 31, 2020

Related U.S. Application Data
(60) Provisional application No. 62/694,427, filed on Jul. 5, 2018.

(51) Int. Cl. A61K 39/005; A61K 31/4168; A61K 31/541; A61K 31/496; C12N 15/86; A61P 33/02

(52) U.S. Cl. CPC A61K 39/005; A61K 31/4168; A61K 31/541; A61K 2039/5154; C12N 15/86; A61P 33/02; A61K 31/496

(57) ABSTRACT

The present disclosure relates to immunogenic formulations and their use for the treatment and prevention of T. cruzi infection, Chagas Disease, and chronic Chagas cardiomyopathy. In certain aspects, methods of producing antigen presenting cells comprising T. cruzi antigens are provided.

The present application claims the priority benefit of U.S. provisional application No. 62/694,427, filed Jul. 5, 2018, the entire contents of which is incorporated herein by reference.

### BACKGROUND OF THE INVENTION

### 1. Field of the Invention

The present invention relates generally to the fields of immunology, infectious disease and medicine. More particularly, it concerns genetically modified dendritic cells and dendritic cell vaccines for the treatment and prevention of intracellular pathogens such as, Chagas disease.

### 2. Description of Related Art

Intracellular pathogens can include viruses, certain types of bacteria as well as more complex organisms such as protozoans. These pathogens cause significant morbidity and mortality world-wide. Importantly, intracellular pathogens can evade certain mechanisms or control the host immune system because at least a portion of their life cycle is with-in the host cell an shielded from some arms of the immune system.

For example, Chagas disease, also known as American Trypanosomiasis, is caused by infection with the protozoan parasite Trypanosoma cruzi (Hotez et al., 2012). It is a leading cause of heart disease in Latin America, with up to 10 million infected people in the Western Hemisphere (Dumonteil et al., 2012). The disease burden of Chagas, based on disability-adjusted life years (DALYs), is five times greater than malaria and approximately one-fifth that of HIV/AIDS in the LAC region (Hotez et al., 2008). Additionally, the annual economic toll for treatment of infected patients exceeds $7 billion globally (Lee et al., 2013). Most of the deaths and disability attributed to Chagas disease result from chronic Chagas cardiomyopathy (CCC) (Rassi et al., 2010) which develops in approximately 30% of infected individuals years to decades after the initial infection due to cascading effects of parasite induced pathologic changes including inflammation, cardiomyocyte hypertrophy, and fibrosis (Bern et al., 2007; Higuchi et al., 2003; Tanowitz et al., 2009). CCC patients develop conduction disturbances associated with arrhythmias and sudden death or end stage characterized by gross enlargement with high right or left ventricular apical aneurysm. Histologically, diffuse and patchy chronic myocarditis with mononuclear cell infiltrates and fibrosis is evident (Biolo et al., 2010; Acquatella, 2007). Two drugs, benznidazole and nifurtimox, have been used for treatment since the 1970s with limited efficacy and significant side effects. Both drugs have up to 100% efficacy in congenital infection when treated within the first years of life, and 65-80% efficacy in children treated during the acute phase. However, adults treated during the chronic phase only achieve 15-35% efficacy (Le Loup et al., 2011; Molina et al., 2014). A recent meta-analysis concluded that these drugs are of questionable efficacy in preventing the onset of Chagasic cardiomyopathy, and almost 20% of patients fail to complete the months-long drug regimen due to significant associated toxicities (Viotti et al., 2009; Pinazo et al., 2010; Issa and Bocchi, 2010). New chemotherapeutics, such as posaconazole, show promise in preclinical testing but have been of limited efficacy in human studies (Molina et al., 2014; Molina et al., 2000). In a recent trial, trypanocidal therapy with benznidazole in patients with established Chagas cardiomyopathy significantly reduced serum parasite detection but did not significantly reduce cardiac clinical deterioration through five years of follow-up (Morillo et al., 2015). Thus there remains an urgent need to develop new therapies including vaccines to achieve sustained parasitological cure and decreased incidence of sudden cardiac death.

Pre-clinical studies have revealed the essential role of antigen specific immune responses, primarily T-cell responses, in control of T. cruzi parasite burden and cardiac disease. Several candidate antigens, including SA85-L1, Tc52, TSA1 and Tc24 have shown efficacy in reducing parasitemia and endpoint cardiac pathology when used therapeutically (Limon-Flores et al., 2010; Quijano-Hernandez and Dumonteil, 2011; Dumonteil et al., 2004; Sanchez-Burgos et al., 2007). This reduction in parasite burden, cardiac disease, and mortality is due to TH1-type T-cell immunity and, in part, to induction of antigen specific CD8+ T cell responses (Limon-Flores et al., 2010; Gupta and Garg, 2013). To date, vaccines that combine Tc24 and TSA-1 have provided the most dramatic reductions in parasite load and cardiac inflammation in pre-clinical experimental systems. Mice vaccinated therapeutically during the acute phase with a combination Tc24/TSA1 DNA vaccine exhibited up to 75% reduction in peak parasitemia, approximately 60% reduction in cardiac parasite burden, and significant reduction in endpoint cardiac pathology in comparison to unvaccinated mice (Limon-Flores et al., 2010; Dumonteil et al., 2004; Sanchez-Burgos et al., 2007). Similarly, mice vaccinated during the chronic phase with a combination vaccine demonstrated up to 80% survival at 180 days post-infection and reduced endpoint cardiac pathology (Dumonteil et al., 2004; Sanchez-Burgos et al., 2007; Pereira et al., 2015). Additionally, in a dog model of Chagas disease, the same therapeutic DNA vaccine construct reduced by 40% the number of dogs that developed cardiac arrhythmias at 50 days post infection (Quijano-Hernandez et al., 2013). Similar studies have shown protective efficacy using the recombinant protein counterparts of these DNA vaccines (Dumonteil et al., 2012; Martinez-Campos et al., 2015; Barry et al., 2016; Lee et al., 2012).

Dendritic cells (DC) are the most important of the professional antigen presenting cells (APCs) that initiate and direct adaptive immune responses. Upon detection of danger signals, DC migrate to local lymph nodes where they induce a variety of immunogenic responses (Banchereau and Steinman, 1998; Medzhitov, 2007; Flores-Romo, 2001; Joffre et al., 2009; Belkaid and Oldenhove, 2008). Signals induced by ligation of pattern recognition receptors, MHC molecules, costimulatory molecules, and inflammatory cytokine receptors activate DC, enabling them to drive immunogenic T-cell responses (Wu and Horuzsko, 2009). The Src homology region 2 (SH2) domain-containing tyrosine phosphatase-1 (SHP-1) is expressed in a wide variety of immune cells where it plays a largely inhibitory role in cell signaling initiated through a range of stimuli (Zhang et al., 2000). SHP-1 inhibition in DC leads to increased proinflammatory cytokine production and activation of Akt, enhancing DC survival. Importantly, mice vaccinated with SHP-1-inhibited DC mount effective immune responses against both melanoma and prostate tumors, demonstrating that SHP-1 is an intrinsic negative regulator of DC activation signaling. Previous work has demonstrated that a dominant negative kinase-inactivated SHP-1 adjuvant (dnSHP) delivered to DC by means of an adenoviral vector can substantially enhance downstream T-cell responses in the context of vaccination (Ramachandran et al., 2011).

Accordingly, a therapeutic immune-stimulating compositions for treatment and prevention of intra-cellular pathogen infections would be desirable. For example, such a composition could provide an alternative intervention to delay or halt the progression of Chagasic cardiomyopathy. To date, however, highly effective immune stimulating compositions have yet to be developed.

### SUMMARY OF THE INVENTION

In one embodiment, the present disclosure provides an antigen presenting cell comprising the recombinant nucleic acid encoding an expression vector for expression of least a first antigen from an intracellular pathogen. In some aspects, the cell is a dendritic cell. In particular aspects, the cell is a human cell. In specific aspects, the cell is a human dendritic cell. In further aspects, the antigen presenting cell further comprises a recombinant antigen from the intracellular pathogen. In some aspects, the antigen presenting cell comprises said expression vector and further comprises a recombinant protein including at least a portion of said first antigen from the intracellular pathogen. In certain aspects, the first antigen is from an intracellular bacteria. In particular aspects, the first antigen is an antigen from Bartonella henselae, Francisella tularensis, Listeria monocytogenes, Salmonella Typhi, Brucella, Legionella, a Mycobacterium, Nocardia, Rhodococcus equi, Yersinia, Chlamydia, Rickettsia or Coxiella. In specific aspects, the first antigen is an antigen from Mycobacterium leprae or Mycobacterium tuberculosis. In other aspects, the first antigen is from a virus. In certain aspects, the first antigen is from a herpes virus or a retrovirus. In specific aspects, the first antigen is from HIV. In other aspects, the first antigen is from a fungus. In certain aspects, the first antigen is from Histoplasma capsulatum, Cryptococcus neoformans or Pneumocystis jirovecii. In other aspects, the first antigen is from a protozoa. In certain aspects, the first antigen is from an Apicomplexan or a Trypanosomatid. In particular aspects, the first antigen is from a Plasmodium spp., Toxoplasma gondii, Cryptosporidium parvum or Leishmania spp. In some aspects, the first antigen is a T. cruzi antigen. In certain aspects, the first antigen is SA85-L1, Tc52, TSA1 or Tc24. In specific aspects, the first antigen is a T. cruzi Tc24 antigen.

In aspects of the embodiments, the cell comprises an expression vector of at least a second antigen from the pathogen. In some aspects, the first antigen and the second antigen are encoded by the same expression vector. In certain aspects, the first antigen and the second antigen are encoded as a fusion protein. In some aspects, the cell comprises an expression vector that encodes a genetic adjuvant. In certain aspects, the genetic adjuvant comprises the coding sequence for constitutively activated AKT (caAKT), inducible MyD88/CD40 (iMC), or dominant-negative SHP-1 (dnSHP). In specific aspects, the genetic adjuvant is dnSHP. In particular aspects, the genetic adjuvant is encoded on the same vector as the first antigen. In certain aspects, the genetic adjuvant is upstream of the sequence encoding the antigen.

In another embodiment, the present disclosure provides an immunogenic composition comprising the antigen-presenting cells of any of any of the aspects or embodiments provided herein, and a pharmaceutically acceptable carrier.

In another embodiment, the present disclosure provides a method for treating or preventing an infection with an intracellular pathogen in a subject comprising administering to the subject an effective amount of the antigen presenting cells of any of the embodiments or aspects provided herein.

In some embodiments, the present disclosure provides a recombinant nucleic acid comprising a sequence encoding at least a first antigen from T. cruzi and a sequence encoding a genetic adjuvant. In some aspects, the antigen is selected from. SA85-L1, Tc52, TSA1 or Tc24. In certain aspects, the nucleic acid sequence encodes at least two antigens from T. cruzi. In particular aspects, the at least two antigens are encoded as a fusion protein. In specific aspects, the at least two antigens comprise Tc24 and TSA1 antigens of T. cruzi. In some aspects, the Tc24 antigen is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In certain aspects, the Tc24 antigen is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2. In some aspects, the TSA1 antigen is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 5. In certain aspects, the TSA1 antigen is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6. In certain aspects, the genetic adjuvant is constitutively activated AKT (caAKT), inducible MyD88/CD40 (iMC), or dominant-negative SHP-1 (dnSHP). In particular aspects, the genetic adjuvant is dnSHP. In specific aspects, dnSHP at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3. In aspects, dnSHP is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4. In some aspects, the sequence encoding a genetic adjuvant is upstream of the sequence encoding the antigen.

In some embodiments, the present disclosure provides an expression vector comprising recombinant nucleic acid comprising a sequence encoding at least a first antigen from T. cruzi and a sequence encoding a genetic adjuvant. In some aspects, the antigen is selected from. SA85-L1, Tc52, TSA1 or Tc24. In certain aspects, the nucleic acid sequence encodes at least two antigens from T. cruzi. In particular aspects, the at least two antigens are encoded as a fusion protein. In specific aspects, the at least two antigens comprise Tc24 and TSA1 antigens of T. cruzi. In some aspects, the Tc24 antigen is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In certain aspects, the Tc24 antigen is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2. In some aspects, the TSA1 antigen is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 5. In certain aspects, the TSA1 antigen is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6. In certain aspects, the genetic adjuvant is constitutively activated AKT (caAKT), inducible MyD88/CD40 (iMC), or dominant-negative SHP-1 (dnSHP). In particular aspects, the genetic adjuvant is dnSHP. In specific aspects, dnSHP at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3. In aspects, dnSHP is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4. In some aspects, the sequence encoding a genetic adjuvant is upstream of the sequence encoding the antigen. In certain aspects, the expression vector is an adenoviral, adeno associated, or retroviral vector. In specific aspects, the expression vector is an adenoviral vector.

In some embodiments, the present disclosure provides a host cell comprising the recombinant nucleic acid or expression vector of any of the above embodiments or aspects. In some aspects, the host cell is an antigen presenting cell. In particular aspects, the cell is a dendritic cell. In certain aspects, the cell is a human cell. In specific aspects, the cell is a human dendritic cell. In some aspects, the antigen presenting cell further comprises a recombinant T. cruzi antigen. In certain aspects, the recombinant T. cruzi antigen comprises a Tc24 protein. In certain aspects, the Tc24 protein comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1.

In some embodiments, the present disclosure provides a composition comprising antigen-presenting cells, wherein the antigen-presenting cells comprise at least a first T. cruzi polypeptide antigen and a genetic adjuvant. In some aspects, the antigen is selected from. SA85-L1, Tc52, TSA1 or Tc24. In certain aspects, the cells comprise at least two antigens from T. cruzi. In particular aspects, the at least two antigens are present as a fusion protein. In specific aspects, the at least two antigens comprise the Tc24 and TSA1 antigens of T. cruzi. In some aspects, the Tc24 antigen is at least 80%, 81%, 82%, 83%, 84%, 85%, 86%, 87%, 88%, 89%, 90%, 91%, 92%, 93%, 94%, 95%, 96%, 97%, 98%, or 99% identical to amino acid sequence of SEQ ID NO: 1. In some aspects, the TSA1 antigen is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 5. In particular aspects, the Tc24 and TSA1 genes are present on an expression vector in said antigen-presenting cells. In certain aspects, the Tc24 nucleotide sequence is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2. In certain aspects, the TSA1 nucleotide sequence is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6. In some aspects, the cells are dendritic cells. In certain aspects, the cells are human cells. In specific aspects, the cells are human dendritic cells. In particular aspects, the antigen-presenting cells are mature dendritic cells. In certain aspects, the antigen presenting cells have been loaded with exogenous recombinant Tc24 protein.

In aspects of the embodiments, the expression vector is an adenoviral vector. In some aspects, the genetic adjuvant is constitutively activated AKT (caAKT), inducible MyD88/CD40 (iMC), or dominant-negative SHP-1 (dnSHP). In specific aspects, the genetic adjuvant is dnSHP. In particular aspects, the dnSHP is a kinase-inactivated SHP1. In specific aspects, the kinase-inactivated SHP1 comprises an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3. In some aspects, the gene encoding the genetic adjuvant is present in the same expression vector as the genes encoding Tc24 and TSA1. In certain aspects, the gene encoding the genetic adjuvant is upstream of the genes encoding Tc24 and TSA1. In other aspects, the gene encoding the genetic adjuvant is downstream of the genes encoding the Tc24 and TSA1 antigens. In specific aspects, the exogenous recombinant Tc24 protein has an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1.

In another embodiment, there is provided an immunogenic composition comprising the antigen-presenting cells of any of the other embodiments or aspects of the disclosure, and a pharmaceutically acceptable carrier. In some embodiments there is provided a method for treating or preventing Chagas disease comprising administering to a subject that has, may have, or is likely to contract Chagas disease a therapeutically effective amount of said immunogenic composition. In some embodiments, there is provided a method for treating or preventing Chagasic cardiomyopathy comprising administering to a patient with Chagas disease a therapeutically effective amount of said immunogenic composition. In some embodiments, there is provided a method for reducing T. cruzi parasite burden in a subject infected with T. cruzi comprising administering to the subject a therapeutically effective amount of said immunogenic composition. In some aspects, the methods further comprise administering at least a second treatment for T. cruzi infection to the subject. In certain aspects, the second treatment is a trypanocidal, chemotherapeutic, or immunotherapeutic treatment. In particular aspects, the trypanocidal treatment is treatment with benznidazole or nifurtimox. In certain aspects, the chemotherapeutic treatment is treatment with posaconazole. In specific aspects, the immunotherapeutic treatment is a second administration of said immunogenic composition. In other aspects, the immunotherapeutic treatment is vaccination with the SA85-L1, Tc52, TSA1, or Tc24 antigens. In some embodiments, there is provided a method for inducing an immune response in a subject comprising administering to the subject a therapeutically effective amount of said immunogenic composition.

In another embodiment, there is provided a method for preparing T. cruzi antigen presenting dendritic cells comprising: (a) obtaining immature antigen presenting cell precursors from a subject; (b) culturing the immature antigen presenting cell precursors to induce differentiation into immature dendritic cells; (c) transducing the immature dendritic cells with a vector for the expression of T. cruzi antigens; (d) contacting the immature dendritic cells with a recombinant T. cruzi antigen; and (e) culturing the immature dendritic cells to produce mature dendritic cells. In some aspects, the T. cruzi antigens encoded by the vector are Tc24 and TSA1. In certain aspects, the Tc24 antigen is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In particular aspects, the Tc24 antigen is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2. In certain aspects, the TSA1 antigen is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO 5. In particular aspects, the TSA1 antigen is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6. In particular aspects, recombinant T. cruzi antigen is recombinant Tc24. In certain aspects, the recombinant Tc24 at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In some aspects, the cells are transduced with the vector and contacted with recombinant T. cruzi antigen together. In other aspects, the cells are transduced with the vector prior to contact with the recombinant T. cruzi antigen. In yet other aspects, the cells are contacted with the recombinant T. cruzi antigen prior to transduction with the vector. In another aspect, the the vector further comprises a gene encoding a genetic adjuvant. In some aspects, the the genetic adjuvant is constitutively activated AKT (caAKT), inducible MyD88/CD40 (iMC), or dominant-negative SHP-1 (dnSHP). In certain aspects, the genetic adjuvant is dnSHP. In particular aspects, the dnSHP is a kinase inactivated dominant negative SHP-1. In specific aspects, the kinase inactivated dominant negative SHP-1 is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3. In specific aspects, the kinase inactivated dominant negative SHP-1 is encoded by a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:4. In some aspects, the vector is a viral vector. In certain aspects, viral vector is an adenoviral, adeno associated, or retroviral vector. In specific aspects, the vector is an adenoviral vector.

In some embodiments, the present disclosure provides a composition comprising the mature dendritic cells produced by the method of any of the embodiments or aspects of the disclosure, and a pharmaceutically acceptable carrier. In some embodiments there is provided a method for treating or preventing Chagas disease comprising administering to a subject that has, may have, or is likely to contract Chagas disease a therapeutically effective amount of said composition. In another embodiment there is provided a method for treating or preventing Chagasic cardiomyopathy comprising administering to a subject with Chagas disease a therapeutically effective amount of said composition. In yet another embodiment, the rei sprovided a method for reducing T. cruzi parasite burden in a subject infected with T. cruzi comprising administering to the subject a therapeutically effective amount of said composition. In some aspects, these methods may further comprise administering at least a second treatment for T. cruzi infection to the subject. In certain aspects, the second treatment is a trypanocidal, chemotherapeutic, or immunotherapeutic treatment. In particular aspects, the trypanocidal treatment is treatment with benznidazole or nifurtimox. In specific aspects, the chemotherapeutic treatment is treatment with posaconazole. In some aspects, the immunotherapeutic treatment is a second administration of said dendritic cell composition. In other aspects, the immunotherapeutic treatment is vaccination with SA85-L1, Tc52, TSA1, or Tc24 antigens. In another embodiment, there is provided a method for inducing an immune response in a subject comprising administering to the subject a therapeutically effective amount of said dendritic cell composition.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C: Bicistronic adenoviral constructs encoding functional adjuvants and parasite specific antigens: Replication-deficient human Type 5 recombinant adenovirus vectors were constructed with genetic adjuvants caAKT (constitutively-activated Akt), iMC (inducible MyD88/CD40), or dnSHP (dominant-negative SHP-1) upstream of the T. cruzi antigens Tc24 and TSA1. (FIG. 1A) HA-tagged genetic adjuvants (Akt, iMC and dnSHP) were cloned into the XbaI site of the pShuttle plasmid driven by CMV promoter. The T. cruzi antigens Tc24 and TSA1 were cloned as a fusion fragment downstream of the adjuvants between NheI and NotI sites. A single glycine hexamer linker separated the antigens while a P2A sequence inserted between antigens and adjuvant permitted cleavage of the two. The expression cassette was cloned into the PmeI site of the pShuttle plasmid. (FIG. 1B) Restriction digestion of ampicillin resistant positive shuttle plasmid clones with PmeI and XbaI generated the Shuttle backbone fragment (4 kb), fragment corresponding to the adjuvant (1.4 kb) and fusion fragment corresponding to the antigens (2.5 kb). The clones were later confirmed with direct sequencing. (FIG. 1C). Psce-I and Iceu-I digestion of the shuttle plasmid released the construct that was then ligated into the adenoviral backbone following manufacturer's instructions. Ampicillin resistant clones were selected and further confirmation of the positive clones was performed by means of Psce-I and Iceu-I restriction digest followed by direct sequencing. Standard 1 kb ladder indicates DNA fragment size with doublet at 2.0 and 1.65 kb.

FIG. 2: Genetic adjuvantation with dNSHP gave significantly enhanced antigen specific IFN-γ responses. Mice were vaccinated with each of the adenoviral constructs (caAKT-Tc24/TSA1, iMC-Tc24/TSA1 and dnSHP-Tc24/TSA1) and antigen specific responses were measured by IFN-γ secretion of splenocytes stimulated with and without 10 μg/mL Tc24 protein by ELISPOT. Compared to caAKT and iMC, genetic adjuvantation with dnSHP gave significant enhancement in the production of Tc24-specific IFN-γ responses.

FIGS. 3A-E: Expression of genetic adjuvant and parasite specific antigens in DCs transduced with adenoviral vector. Low titer adenovirus was made with dnSHP as genetic adjuvant. (FIGS. 2A-D) 293 T cells and Dendritic cells were transduced with adenovirus expressing dNSHP (genetic adjuvant) and Tc24 and TSA1 (antigens). Western blots of cell lysates were probed with anti-HA, anti-SHP, anti-Tc24 and anti-TSA1 antibodies to confirm the expression of adjuvant, antigen and fusion protein products. (FIG. 3E) Dendritic cells were transduced with different titrations of the viral particles and cell lysates were probed with anti-SHP1 antibody. Doses as low as 100 (10²) vp per cell gave good expression of the adjuvant as confirmed by western blots. Protein molecular weights (in kDa) are indicated on the left-hand side of each blot.

FIGS. 4A-E: DC vaccines transduced with dnSHP adenoviral construct and loaded with Tc24 recombinant protein reduce T. cruzi parasite burden. (FIG. 4A) To test therapeutic efficacy of the vaccine, naïve Balb/c mice were infected intraperitoneally with 500 blood form trypomastigotes. Seven days post-infection (dpi) the mice were therapeutically immunized intraperitoneally with 250,000 DC transduced with the dnSHP vector alone, DC loaded with only Tc24 recombinant protein, DC both transduced with the dnSHP vector and simultaneously loaded with Tc24 recombinant protein. Unloaded DC served as controls. Boost vaccination was given 14 dpi. At 50 dpi, all remaining mice were sacrificed and analyzed. (FIG. 4B) Serum antibody titers analyzed from the mice at end of the study demonstrated that mice treated with vector+protein-loaded DC exhibited significantly elevated Tc24-specific IgG1 antibody titers (p<0.004). (FIG. 4C) End point cardiac parasite DNA was quantified by qPCR analysis using T. cruzi specific primers. A trend toward low/no detectable parasite DNA (p=0.08) was observed among mice that received vector+protein loaded DC compared to the other groups. (FIGS. 4D-E) H&E staining was performed on cardiac tissue at the end of the study and pathological scores on a scale of 0 to 5 were given for lymphocytic infiltration with 5 being the maximum infiltration. Randomized and blinded pathological scoring demonstrated that mice which received vector+protein loaded DC exhibited substantially lower pathological index scores, some on par with those of uninfected mice (p<0.0006). End point cardiac size (enlargement) as assessed by cross-sectional distance was nearly 20% less (5.04 mm vs 6.16 mm, p<0.0001) among mice that received vector+protein loaded DC and similar to that of uninfected mice. For all panels error bars=+/−SEM.

FIGS. 5A-0: DC vaccines transduced with dnSHP adenoviral construct and loaded with Tc24 recombinant protein improve cardiac pathology. (FIGS. 5A-E) Representative end-point cardiac H&E histopathology reveals amastigote nest formation in mice from all groups except the ones treated with DC loaded with vector+protein. Images shown at 100× magnification. Scale bar=20 μm. (FIGS. 5F-J) Representative H&E images of the amastigote nests in cardiac tissue of each group of mice. Images shown at 400× magnification. Scale bar=5 μm. (FIGS. 5K-O) Representative Masson's Trichrome stained images showing the extent of cardiac inflammatory fibrosis among mice of each treated group. Images shown at 100× magnification. Scale bar=20 μm.

FIG. 6: Quantitation of Masson's Trichrome staining. To quantify cardiac fibrosis 5 μm sections of heart tissue were stained with Masson's Trichrome stain. Images of three to five representative sections from each mouse were captured at 100× magnification using a Fisher Micromaster microscope and Micron software. Images were evaluated by a reviewer blinded to the treatment groups and analyzed using ImageJ FIJI software to quantify the area of fibrosis and total tissue area. Fibrosis in the vector+protein group was significantly lower than all other infected groups as determined by one-way ANOVA (**p<0.01). Analysis of uninfected age-matched control mice is shown on the far right for comparison. Error bars=+/−SEM.

FIGS. 7A-C: Survival curves and blood form parasitemia. (FIG. 7A) Kaplan-Meier survival analysis of all infected groups indicated no significant differences in overall survival between day 0 (infection) and day 50 (sacrifice) as determined by log-rank (Mantle-Cox). (FIG. 7B) Total blood form parasitemia of each experimental group was quantitated by PCR of serial bleeds taken on post-infection days 7, 10, 14, 17, 21, 24, 28, 31, and 43. No statistically significant differences were observed between groups as determined by Kruskal-Wallis ANOVA with Dunn's multiple comparisons test. Y axis=parasite equivalents/ml whole blood. (FIG. 7C) Area under the curve (AUC) analysis of each individual animal's parasitemia curve also indicated no statistically significant differences in blood form parasitemia between groups. Y axis=parasite equivalents/ml whole blood. Experimental cohorts consisted of n=4-5 animals each.

### DESCRIPTION OF ILLUSTRATIVE EMBODIMENTS

Intracellular infections possess unique characteristics that can make them challenging to treat and vaccinate against. In particular, because at least a portion of the pathogen's life cycle is shielded from certain immunes responses with-in the host cell, not all types of immune system attack are effective to control in the infection. In particular, it would be desirable to provide an immune stimulating composition that could specifically direct immune cells, such as cytotoxic T-cells, to effectively target infected host cells. Studies herein demonstrate a system to provide just such a infected host cell-targeted immune response.

One example of an intracellular pathogen infection is Chagas disease. Chagas is a major neglected tropical disease caused by persistent chronic infection with the protozoan parasite Trypanosoma cruzi. An estimated 11 million people are infected with T. cruzi, however only two drugs are approved for treatment and no vaccines are available. Chagas disease is also a leading cause of heart disease in Latin America. Thus there is an urgent need to develop vaccines and new drugs to prevent and treat Chagas disease and cardiomyopathy associated with Chagas disease, such as chronic Chagas cardiomyopathy. In this work, the inventors have prepared an immunogenic dendritic cell vaccine for the treatment of Chagas disease in which the dendritic cells have been loaded with recombinant Tc24 protein and transduced with an adenoviral vector comprising genes encoding a dominant negative kinase-inactivated SHP1 protein as well as the Tc24 and antigens from T. cruzi. This vaccine was used to treat mice infected with T. cruzi and shown to generate an effective antigen-specific immune response, decrease parasite burden, and mitigate Chagas associated cardiomyopathy. These and other aspects of the disclosure are described in detail below.

### I. Definitions

As used herein, the term “antigen” is a molecule capable of being bound by an antibody or T-cell receptor. An antigen is additionally capable of inducing a humoral immune response and/or cellular immune response leading to the production of B- and/or T-lymphocytes. The structural aspect of an antigen that gives rise to a biological response is referred to herein as an “antigenic determinant.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another.

An “epitope,” also known as an antigenic determinant, is the part of a macromolecule that is recognized by the immune system, specifically by antibodies, B cells, or T cells. The terms “inhibiting,” “reducing,” or “prevention,” or any variation of these terms, when used in the claims and/or the specification includes any measurable decrease or complete inhibition to achieve a desired result.

As used herein, an “amino acid” or “amino acid residue” refers to any naturally-occurring amino acid, any amino acid derivative or any amino acid mimic known in the art, including modified or unusual amino acids. In certain embodiments, the natural residues of the peptide are sequential, without any non-amino acid interrupting the sequence of natural amino acid residues. In other embodiments, the sequence may comprise one or more non-natural amino acid moieties.

The term “peptide” is used interchangeably with “oligopeptide” in the present specification to designate a series of residues, typically L-amino acids, connected one to the other, typically by peptide bonds between the α-amino and carboxyl groups of adjacent amino acids. Particular T cell-inducing oligopeptides of the disclosure are 15 residues or less in length and usually consist of between about 8 and about 13 residues, particularly 9 to 11 residues. Specific lengths of 9, 10, 11, 12, 13, 14 and 15 residues are contemplated.

An “immunogenic peptide” or “peptide epitope” is a peptide which comprises an allele-specific motif or supermotif such that the peptide will bind an HLA molecule and induce a T cell response. Thus, immunogenic peptides of the disclosure are capable of binding to an appropriate HLA molecule and thereafter inducing a T cell response to the antigen from which the immunogenic peptide is derived.

As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In particular embodiments, biocompatible protein, polypeptide or peptide containing compositions will generally be mammalian proteins or peptides or synthetic proteins or peptides each essentially free from toxins, pathogens and harmful immunogens.

Major histocompatibility complex” or “MHC” is a cluster of genes that plays a role in control of the cellular interactions responsible for physiologic immune responses. In humans, the MHC complex is also known as the HLA complex. For a detailed description of the MHC and HLA complexes (see Paul, 1993).

A “protective immune response” refers to a T cell response to an antigen derived from an infectious agent, which prevents or at least partially arrests disease symptoms or infection. The immune response may also include an antibody response which has been facilitated by the stimulation of helper T cells.

As used herein, “carrier” includes any and all solvents, dispersion media, vehicles, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous injectable composition that contains a protein as an active ingredient is well understood in the art.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### II. Chagas Disease

### A. Background

Chagas disease, or American trypanosomiasis, is a tropical parasitic disease caused by the protozoan Trypanosoma cruzi. It is spread mostly by insects known as triatominae or kissing bugs. The symptoms change over the course of the infection. In the early stage, symptoms are typically either not present or mild and may include fever, swollen lymph nodes, headaches, or local swelling at the site of the bite. After 8-12 weeks, individuals enter the chronic phase of disease and in 60-70% it never produces further symptoms. The other 30 to 40% of people develop further symptoms 10 to 30 years after the initial infection, including enlargement of the ventricles of the heart in 20 to 30%, leading to heart failure. An enlarged esophagus or an enlarged colon may also occur in 10% of people.

T. cruzi is commonly spread to humans and other mammals by the blood-sucking “kissing bugs” of the subfamily Triatominae. These insects are known by a number of local names, including: vinchuca in Argentina, Bolivia, Chile and Paraguay, barbeiro (the barber) in Brazil, pito in Colombia, chinche in Central America, and chipo in Venezuela. The disease may also be spread through blood transfusion, organ transplantation, eating food contaminated with the parasites, and by vertical transmission (from a mother to her fetus). Diagnosis of early disease is by finding the parasite in the blood using a microscope. Chronic disease is diagnosed by finding antibodies for T. cruzi in the blood.

Prevention mostly involves eliminating kissing bugs and avoiding their bites. Other preventative efforts include screening blood used for transfusions. A vaccine has not been developed as of 2013. Early infections are treatable with the medication benznidazole or nifurtimox. Medication nearly always results in a cure if given early, but becomes less effective the longer a person has had Chagas disease. When used in chronic disease, medication may delay or prevent the development of end-stage symptoms. Benznidazole and nifurtimox cause temporary side effects in up to 40% of people including skin disorders, brain toxicity, and digestive system irritation.

It is estimated that 7 to 8 million people, mostly in Mexico, Central America and South America, have Chagas disease as of 2013. In 2006, Chagas was estimated to result in 12,500 deaths per year. Most people with the disease are poor, and most people with the disease do not realize they are infected. Large-scale population movements have increased the areas where Chagas disease is found and these include many European countries and the United States. These areas have also seen an increase in the years up to 2014. The disease was first described in 1909 by Carlos Chagas after whom it is named. It affects more than 150 other animals.

### B. T. cruzi

The Trypanosoma cruzi life cycle starts in an animal reservoir, usually mammals, wild or domestic, including humans. A triatomine (Triatoma infestans) insect serves as the vector. While feeding, the traitomine insect may ingest T. cruzi. Once ingested by the triatomine insect, the parasite goes into the epimastigote stage, making it possible to reproduce. After reproducing through binary fission, the epimastigotes move onto the rectal cell wall, where they become infectious. Infectious T. cruzi are referred to as metacyclic trypomastigotes. When the triatomine insect subsequently feeds, it defecates and the trypomastigotes in the feces may swim into the host's cells using flagella, a characteristic swimming tail dominant in the Euglenoid class of protists.

The trypomastigotes then enter the human host through the bite wound or by crossing mucous membranes. The host cells contain macromolecules such as laminin, thrombospondin, heparin sulphate, and fibronectin that cover their surface. These macromolecules are essential for adhesion between the parasite and host, and for the process of host invasion by the parasite. The trypomastigotes must cross a network of proteins that line the exterior of the host cells in order to make contact and invade the host cells. The molecules and proteins on the cytoskeleton of the cell also bind to the surface of the parasite and initiate host invasion. When they enter a human cell, they enter a second reproductive stage, becoming amastigotes. After reproducing through binary fission until a large amount of amastigotes is present in a cell, pseudocysts are formed, the amastigotes then turn back into trypomastigotes, and the cell bursts. The newly released trypomastigotes then swim along to either infect other cells or are consumed by other reduviid insects.

### C. Symptoms

The human disease occurs in two stages: an acute stage, which occurs shortly after an initial infection, and a chronic stage that develops over many years. The acute phase lasts for the first few weeks or months of infection. It usually occurs unnoticed because it is symptom-free or exhibits only mild symptoms that are not unique to Chagas disease. These can include fever, fatigue, body aches, muscle pain, headache, rash, loss of appetite, diarrhea, nausea, and vomiting. The signs on physical examination can include mild enlargement of the liver or spleen, swollen glands, and local swelling (a chagoma) where the parasite entered the body.

The most recognized marker of acute Chagas disease is called Romaria's sign, which includes swelling of the eyelids on the side of the face near the bite wound or where the bug feces were deposited or accidentally rubbed into the eye. Rarely, young children, or adults may die from the acute disease due to severe inflammation/infection of the heart muscle (myocarditis) or brain (meningoencephalitis). The acute phase also can be severe in people with weakened immune systems.

If symptoms develop during the acute phase, they usually resolve spontaneously within three to eight weeks in approximately 90% of individuals. Although the symptoms resolve, even with treatment the infection persists and enters a chronic phase. Of individuals with chronic Chagas disease, 60-80% will never develop symptoms (called indeterminate chronic Chagas disease), while the remaining 20-40% will develop life-threatening heart and/or digestive disorders during their lifetime (called determinate chronic Chagas disease). In 10% of individuals, the disease progresses directly from the acute form to a symptomatic clinical form of chronic Chagas disease.

The symptomatic (determinate) chronic stage affects the nervous system, digestive system and heart. About two-thirds of people with chronic symptoms have cardiac damage, including dilated cardiomyopathy, which causes heart rhythm abnormalities and may result in sudden death. About one-third of patients go on to develop digestive system damage, resulting in dilation of the digestive tract (megacolon and megaesophagus), accompanied by severe weight loss. Swallowing difficulties (secondary achalasia) may be the first symptom of digestive disturbances and may lead to malnutrition.

20% to 50% of individuals with intestinal involvement also exhibit cardiac involvement. Up to 10% of chronically infected individuals develop neuritis that results in altered tendon reflexes and sensory impairment. Isolated cases exhibit central nervous system involvement, including dementia, confusion, chronic encephalopathy and sensory and motor deficits.

The clinical manifestations of Chagas disease are due to cell death in the target tissues that occurs during the infective cycle, by sequentially inducing an inflammatory response, cellular lesions, and fibrosis. For example, intracellular amastigotes destroy the intramural neurons of the autonomic nervous system in the intestine and heart, leading to megaintestine and heart aneurysms, respectively. If left untreated, Chagas disease can be fatal, in most cases due to heart muscle damage.

### D. Transmission

In Chagas-endemic areas, the main mode of transmission is through an insect vector called a triatomine bug. A triatomine becomes infected with T. cruzi by feeding on the blood of an infected person or animal. During the day, triatomines hide in crevices in the walls and roofs.

The bugs emerge at night, when the inhabitants are sleeping. Because they tend to feed on people's faces, triatomine bugs are also known as “kissing bugs”. After they bite and ingest blood, they defecate on the person. Triatomines pass T. cruzi parasites (called trypomastigotes) in feces left near the site of the bite wound.

Scratching the site of the bite causes the trypomastigotes to enter the host through the wound, or through intact mucous membranes, such as the conjunctiva. Once inside the host, the trypomastigotes invade cells, where they differentiate into intracellular amastigotes. The amastigotes multiply by binary fission and differentiate into trypomastigotes, which are then released into the bloodstream. This cycle is repeated in each newly infected cell. Replication resumes only when the parasites enter another cell or are ingested by another vector.

Dense vegetation (such as that of tropical rainforests) and urban habitats are not ideal for the establishment of the human transmission cycle. However, in regions where the sylvatic habitat and its fauna are thinned by economic exploitation and human habitation, such as in newly deforested areas, piassava palm culture areas, and some parts of the Amazon region, a human transmission cycle may develop as the insects search for new food sources.

T. cruzi can also be transmitted through blood transfusions. With the exception of blood derivatives (such as fractionated antibodies), all blood components are infective. The parasite remains viable at 4° C. for at least 18 days or up to 250 days when kept at room temperature. It is unclear whether T. cruzi can be transmitted through frozen-thawed blood components.

Other modes of transmission include organ transplantation, through breast milk, and by accidental laboratory exposure. Chagas disease can also be spread congenitally (from a pregnant woman to her baby) through the placenta, and accounts for approximately 13% of stillborn deaths in parts of Brazil.

Oral transmission is an unusual route of infection, but has been described. In 1991, farm workers in the state of Paraiba, Brazil, were infected by eating contaminated food; transmission has also occurred via contaminated acai palm fruit juice and garapa. A 2007 outbreak in 103 Venezuelan school children was attributed to contaminated guava juice.

Chagas disease is a growing problem in Europe, because the majority of cases with chronic infection are asymptomatic and because of migration from Latin America.

### E. Diagnosis

The presence of T. cruzi is diagnostic of Chagas disease. It can be detected by microscopic examination of fresh anticoagulated blood, or its buffy coat, for motile parasites; or by preparation of thin and thick blood smears stained with Giemsa, for direct visualization of parasites. Microscopically, T. cruzi can be confused with Trypanosoma rangeli, which is not known to be pathogenic in humans. Isolation of T. cruzi can occur by inoculation into mice, by culture in specialized media (for example, NNN, LIT); and by xenodiagnosis, where uninfected Reduviidae bugs are fed on the patient's blood, and their gut contents examined for parasites.

Various immunoassays for T. cruzi are available and can be used to distinguish among strains (zymodemes of T. cruzi with divergent pathogenicities). These tests include: detecting complement fixation, indirect hemagglutination, indirect fluorescence assays, radioimmunoassays, and ELISA. Alternatively, diagnosis and strain identification can be made using polymerase chain reaction (PCR).

### F. Prevention

There is currently no vaccine against Chagas disease. Prevention is generally focused on decreasing the numbers of the insect that spreads it (Triatoma) and decreasing their contact with humans. This is done by using sprays and paints containing insecticides (synthetic pyrethroids), and improving housing and sanitary conditions in rural areas. For urban dwellers, spending vacations and camping out in the wilderness or sleeping at hostels or mud houses in endemic areas can be dangerous; a mosquito net is recommended. Some measures of vector control include:

A yeast trap can be used for monitoring infestations of certain species of triatomine bugs (Triatoma sordida, Triatoma brasiliensis, Triatoma pseudomaculata, and Panstrongylus megistus). Promising results were gained with the treatment of vector habitats with the fungus Beauveria bassiana. Targeting the symbionts of Triatominae through paratransgenesis can be performed.

A number of potential vaccines are currently being tested. Vaccination with Trypanosoma rangeli has produced positive results in animal models. More recently, the potential of DNA vaccines for immunotherapy of acute and chronic Chagas disease is being tested by several research groups.

Blood transfusion was formerly the second-most common mode of transmission for Chagas disease, but the development and implementation of blood bank screening tests has dramatically reduced this risk in the 21st century. Blood donations in all endemic Latin American countries undergo Chagas screening, and testing is expanding in countries, such as France, Spain and the United States, that have significant or growing populations of immigrants from endemic areas. In Spain, donors are evaluated with a questionnaire to identify individuals at risk of Chagas exposure for screening tests.

The US FDA has approved two Chagas tests, including one approved in April 2010, and has published guidelines that recommend testing of all donated blood and tissue products. While these tests are not required in U.S., an estimated 75-90% of the blood supply is currently tested for Chagas, including all units collected by the American Red Cross, which accounts for 40% of the U.S. blood supply. The Chagas Biovigilance Network reports current incidents of Chagas-positive blood products in the United States, as reported by labs using the screening test approved by the FDA in 2007.

### G. Treatment

There are two approaches to treating Chagas disease, antiparasitic treatment, to kill the parasite; and symptomatic treatment, to manage the symptoms and signs of the infection. Management uniquely involves addressing selective incremental failure of the parasympathetic nervous system. Autonomic disease imparted by Chagas may eventually result in megaesophagus, megacolon and accelerated dilated cardiomyopathy. The mechanisms that explain why Chagas targets the parasympathetic autonomic nervous system, and spares the sympathetic autonomic nervous system, remain poorly understood.

1. Medication

Antiparasitic treatment is most effective early in the course of infection, but is not limited to cases in the acute phase. Drugs of choice include azole or nitro derivatives, such as benznidazole or nifurtimox. Both agents are limited in their capacity to effect parasitologic cure (a complete elimination of T. cruzi from the body), especially in chronically infected patients, and resistance to these drugs has been reported.

Studies suggest antiparasitic treatment leads to parasitological cure in about 60-85% of adults and more than 90% of infants treated in the first year of acute phase Chagas disease. Children (aged six to 12 years) with chronic disease have a cure rate of about 60% with benznidazole. While the rate of cure declines the longer an adult has been infected with Chagas, treatment with benznidazole has been shown to slow the onset of heart disease in adults with chronic Chagas infections.

Treatment of chronic infection in women prior to or during pregnancy does not appear to reduce the probability the disease will be passed on to the infant. Likewise, it is unclear whether prophylactic treatment of chronic infection is beneficial in persons who will undergo immunosuppression (for example, organ transplant recipients) or in persons who are already immunosuppressed (for example, those with HIV infection).

2. Complications

In the chronic stage, treatment involves managing the clinical manifestations of the disease. For example, pacemakers and medications for irregular heartbeats, such as the anti-arrhythmia drug amiodarone, may be life saving for some patients with chronic cardiac disease, while surgery may be required for megaintestine. The disease cannot be cured in this phase, however. Chronic heart disease caused by Chagas disease is now a common reason for heart transplantation surgery. Until recently, however, Chagas disease was considered a contraindication for the procedure, since the heart damage could recur as the parasite was expected to seize the opportunity provided by the immunosuppression that follows surgery.

It was noted that survival rates in Chagas patients could be significantly improved by using lower dosages of the immunosuppressant drug cyclosporin. Recently, direct stem cell therapy of the heart muscle using bone marrow cell transplantation has been shown to dramatically reduce risks of heart failure in Chagas patients.

### III. Dendritic Cell Vaccines

In one embodiment of the invention, a vaccine comprising antigen presenting cells comprising T. cruzi antigens and a genetic adjuvant are disclosed. Methods for the production of dendritic cells for vaccines can be found in, e.g., U.S. Pat. No. 8,728,806, incorporated herein by reference, in its entirety. In aspects of the disclosure, the vaccine is a composition of dendritic cells comprising the Tc24 and TSA1 proteins of T. cruzi and a genetic adjuvant, such as dominant negative kinase-deficient SHP1. In some aspects, the Tc24, TSA1, and genetic adjuvant are encoded by a vector, such as an expression vector, or a viral vector, such as an adenoviral vector. In some embodiments, the dendritic cells are also loaded with exogenous recombinant Tc24 protein.

A. Dendritic Cells

A “dendritic cell” (DC) belongs to a group of cells called professional antigen presenting cells (APCs). DCs have a characteristic morphology, with thin sheets (lamellipodia) extending from the dendritic cell body in several directions. Several phenotypic criteria are also typical, but can vary depending on the source of the dendritic cell. These include high levels of MHC molecules (e.g., class I and class II MHC) and costimulatory molecules (e.g., B7-1 and B7-2), and a lack of markers specific for granulocytes, NK cells, B cells, and T cells. Many dendritic cells express certain markers; for example, some Human dendritic cells selectively express CD83, a member of the immunoglobulin superfamily (Zhou and Tedder (1995) Journal of Immunology 3821-3835). Dendritic cells are able to initiate primary T cell responses in vitro and in vivo. These responses are antigen specific. Dendritic cells direct a strong mixed leukocyte reaction (MLR) compared to peripheral blood leukocytes, splenocytes, B cells and monocytes. Dendritic cells are optionally characterized by the pattern of cytokine expression by the cell (Zhou and Tedder (1995) Blood 3295-3301). DCs can be generated in vivo or in vitro from immature precursors (e.g., monocytes).

Methods for isolating cell populations enriched for dendritic cell precursors and immature dendritic cells from various sources, including blood and bone marrow, are known in the art. For example, dendritic cell precursors and immature dendritic cells can be isolated by collecting heparinized blood, by apheresis or leukapheresis, by preparation of buffy coats, rosetting, centrifugation, density gradient centrifugation (e.g., using Ficoll (such as FICOLL-PAQUE®), PERCOLL® (colloidal silica particles (15-30 mm diameter) coated with non-dialyzable polyvinylpyrrolidone (PVP)), sucrose, and the like), differential lysis of cells, filtration, and the like. In certain embodiments, a leukocyte population can be prepared, such as, for example, by collecting blood from a subject, defribrinating to remove the platelets and lysing the red blood cells. Dendritic cell precursors and immature dendritic cells can optionally be enriched for monocytic dendritic cell precursors by, for example, centrifugation through a PERCOLL® gradient. In other aspects, dendritic cell precursors can be selected using CD14 selection of G-CSF mobilized peripheral blood.

Dendritic cell precursors and immature dendritic cells optionally can be prepared in a closed, aseptic system. As used herein, the terms “closed, aseptic system” or “closed system” refer to a system in which exposure to non-sterilize, ambient, or circulating air or other non-sterile conditions is minimized or eliminated. Closed systems for isolating dendritic cell precursors and immature dendritic cells generally exclude density gradient centrifugation in open top tubes, open air transfer of cells, culture of cells in tissue culture plates or unsealed flasks, and the like. In a typical embodiment, the closed system allows aseptic transfer of the dendritic cell precursors and immature dendritic cells from an initial collection vessel to a sealable tissue culture vessel without exposure to non-sterile air.

In certain embodiments, monocytic dendritic cell precursors are isolated by adherence to a monocyte-binding substrate. For example, a population of leukocytes (e.g., isolated by leukapheresis) can be contacted with a monocytic dendritic cell precursor adhering substrate. When the population of leukocytes is contacted with the substrate, the monocytic dendritic cell precursors in the leukocyte population preferentially adhere to the substrate. Other leukocytes (including other potential dendritic cell precursors) exhibit reduced binding affinity to the substrate, thereby allowing the monocytic dendritic cell precursors to be preferentially enriched on the surface of the substrate.

Suitable substrates include, for example, those having a large surface area to volume ratio. Such substrates can be, for example, a particulate or fibrous substrate. Suitable particulate substrates include, for example, glass particles, plastic particles, glass-coated plastic particles, glass-coated polystyrene particles, and other beads suitable for protein absorption. Suitable fibrous substrates include microcapillary tubes and microvillous membrane. The particulate or fibrous substrate usually allows the adhered monocytic dendritic cell precursors to be eluted without substantially reducing the viability of the adhered cells. A particulate or fibrous substrate can be substantially non-porous to facilitate elution of monocytic dendritic cell precursors or dendritic cells from the substrate. A “substantially non-porous” substrate is a substrate in which at least a majority of pores present in the substrate are smaller than the cells to minimize entrapping cells in the substrate.

Adherence of the monocytic dendritic cell precursors to the substrate can optionally be enhanced by addition of binding media. Suitable binding media include monocytic dendritic cell precursor culture media (e.g., AIM-V®, RPMI 1640, DMEM, X-VIVO 15®, and the like) supplemented, individually or in any combination, with for example, cytokines (e.g., Granulocyte/Macrophage Colony Stimulating Factor (GM-CSF), Interleukin 4 (IL-4), or Interleukin 13 (IL-13)), blood plasma, serum (e.g., human serum, such as autologous or allogenic sera), purified proteins, such as serum albumin, divalent cations (e.g., calcium and/or magnesium ions) and other molecules that aid in the specific adherence of monocytic dendritic cell precursors to the substrate, or that prevent adherence of non-monocytic dendritic cell precursors to the substrate. In certain embodiments, the blood plasma or serum can be heated-inactivated. The heat-inactivated plasma can be autologous or heterologous to the leukocytes.

Following adherence of monocytic dendritic cell precursors to the substrate, the non-adhering leukocytes are separated from the monocytic dendritic cell precursor/substrate complexes. Any suitable means can be used to separate the non-adhering cells from the complexes. For example, the mixture of the non-adhering leukocytes and the complexes can be allowed to settle, and the non-adhering leukocytes and media decanted or drained. Alternatively, the mixture can be centrifuged, and the supernatant containing the non-adhering leukocytes decanted or drained from the pelleted complexes.

Isolated dendritic cell precursors can be cultured ex vivo for differentiation, maturation and/or expansion. (As used herein, isolated immature dendritic cells, dendritic cell precursors, T cells, and other cells, refers to cells that, by human hand, exists apart from their native environment, and are therefore not a product of nature. Isolated cells can exist in purified form, in semi-purified form, or in a non-native environment.) Briefly, ex vivo differentiation typically involves culturing dendritic cell precursors, or populations of cells having dendritic cell precursors, in the presence of one or more differentiation agents. Suitable differentiating agents can be, for example, cellular growth factors (e.g., cytokines such as (GM-CSF), Interleukin 4 (IL-4), Interleukin 13 (IL-13), and/or combinations thereof). In certain embodiments, the monocytic dendritic cells precursors are differentiated to form monocyte-derived immature dendritic cells.

The dendritic cell precursors can be cultured and differentiated in suitable culture conditions. Suitable tissue culture media include AIM-V®, RPMI 1640, DMEM, X-VIVO 15®, and the like. The tissue culture media can be supplemented with serum, amino acids, vitamins, cytokines, such as GM-CSF and/or IL-4, divalent cations, and the like, to promote differentiation of the cells. In certain embodiments, the dendritic cell precursors can be cultured in the serum-free media. Such culture conditions can optionally exclude any animal-derived products. A typical cytokine combination in a typical dendritic cell culture medium is about 500 units/ml each of GM-CSF (50 ng/ml) and IL-4 (10 ng/ml). Dendritic cell precursors, when differentiated to form immature dendritic cells, are phenotypically similar to skin Langerhans cells. Immature dendritic cells typically are CD14⁻ and CD11c⁺, express low levels of CD86 and CD83, and are able to capture soluble antigens via specialized endocytosis. The immature DC expressed very high levels of CD86. Also, the population was mixed in terms of CD14 and CD11C. Though the majority were CD11c+, there were distinct subpopulations that were CD11c− and CD 14+.

The immature dendritic cells are matured to form mature dendritic cells. Mature DC lose the ability to take up antigen and display up-regulated expression of costimulatory cell surface molecules and various cytokines. Specifically, mature DC express higher levels of MHC class I and II antigens than immature dendritic cells, and mature dendritic cells are generally identified as being CD80+, CD83+, CD86+, and CD14⁻. Greater MHC expression leads to an increase in antigen density on the DC surface, while up regulation of costimulatory molecules CD80 and CD86 strengthens the T cell activation signal through the counterparts of the costimulatory molecules, such as CD28 on the T cells.

Mature dendritic cells of the present invention can be prepared (i.e., matured) by contacting the immature dendritic cells with effective amounts or concentrations of a nucleic acid composition and a tumor antigen composition. Effective amounts of nucleic acid composition typically range from at most, at least, or about 0.01, 0.1, 1, 5, 10, to 10, 15, 20, 50, 100 ng or mg of nucleic acid per culture dish or per cell, including all values and ranges there between. Effective amounts of tumor antigen composition typically range from at most, at least, or about 0.01, 0.1, 1, 5, 10, to 10, 15, 20, 50, 100 ng or mg of protein per culture dish or per cell. In certain aspects 0.001 ng of tumor antigen/cell to 1 μg of tumor antigen/million cells) can be used. The tumor antigen composition can optionally be heat inactivated or treated (e.g., exposed to protease) prior to contact with dendritic cells. Maturing the immature dendritic cells with a nucleic acid composition and a tumor antigen composition primes the mature dendritic cells for a type 1 (Th-1) response.

The immature DC are typically contacted with effective amounts of a nucleic acid composition and a T. cruzi antigen composition for at most, at least, or about 1, 2, 3, 4, 5, 6, 7, 8, 9, 10, 11, 12, to 10, 11, 12, 13, 14, 15, 16, 17, 18, 19, 20, 21, 22, 23, or 24 minutes, 1, 2, 3, 4, 5, 6, 7, 8, 9, 10, 11, 12, to 10, 11, 12, 13, 14, 15, 16, 17, 18, 19, 20, 21, 22, 23, or 24 hours, or 1, 2, 3, 4, 5, 6, 7, 8, 9, 10, 11, 12, to 10, 11, 12, 13, 14, 15, 16, 17, 18, 19, 20, 21, 22, 23, or 24 days. The immature dendritic cells can be cultured and matured in suitable maturation culture conditions. Suitable tissue culture media include AIM-V®, RPMI 1640, DMEM, X-VIVO 15®, and the like. The tissue culture media can be supplemented with amino acids, vitamins, cytokines, such as GM-CSF and/or IL-4, divalent cations, and the like, to promote maturation of the cells.

Maturation of dendritic cells can be monitored by methods known in the art. Cell surface markers can be detected in assays familiar to the art, such as flow cytometry, immunohistochemistry, and the like. The cells can also be monitored for cytokine production (e.g., by ELISA, FACS, or other immune assay). Dendritic cell precursors, immature dendritic cells, and mature dendritic cells, either primed or unprimed, with antigens can be cryopreserved for use at a later date. Methods for cryopreservation are well-known in the art. For example, U.S. Pat. No. 5,788,963, which is incorporated herein by reference in its entirety.

B. Nucleic Acid Vectors

In some embodiments, vectors can be used to introduce polynucleotide sequences that encode all or part of functional T. cruzi antigens, as well as genetic adjuvants, into a packaging cell line for the preparation of a recombinant virus. In addition to the elements as described herein, the vectors can contain polynucleotide sequences encoding the various components of the recombinant virus and at least one variable region as described herein, as well as any components necessary for the production of the virus that are not provided by the packaging cell line. In other embodiments, in addition to the elements as described herein, the vectors can contain polynucleotide sequences encoding the various components of the recombinant virus and at least one variable region as described here, as well as any components necessary for the production of the virus that are not provided by the packaging cell line. Eukaryotic cell expression vectors are well known in the art and are available from a number of commercial sources.

In some embodiments, one or more multicistronic expression vectors are utilized that include two or more of the elements (e.g., the viral genes, at least one of: Tc24 sequence or TSA1 sequence, a genetic adjuvant such as dnSHP1) necessary for production of a desired recombinant virus in packaging cells. The use of multicistronic vectors reduces the total number of vectors required and thus avoids the possible difficulties associated with coordinating expression from multiple vectors. In a multicistronic vector the various elements to be expressed are operably linked to one or more promoters (and other expression control elements as necessary). In some embodiments a multicistronic vector comprising viral elements and nucleotide sequences encoding all or part of Tc24, TSA1, and a genetic adjuvant, is used.

Each component to be expressed in a multicistronic expression vector may be separated, for example, by an IRES element or a viral 2A element, to allow for separate expression of the various proteins from the same promoter. IRES elements and 2A elements are known in the art (U.S. Pat. No. 4,937,190; de Felipe et al., 2004. Traffic 5: 616-626, each of which is incorporated herein by reference in its entirety). The efficacy of a particular multicistronic vector for use in synthesizing the desired recombinant virus can readily be tested by detecting expression of each of the genes using standard protocols. Exemplary protocols that are well known in the art include, but are not limited to, antibody-specific immunoassays such as Western blotting.

Vectors will usually contain a promoter that is recognized by the packaging cell and that is operably linked to the polynucleotide(s) encoding the antigens, genetic adjuvant, viral components, and the like. A promoter is an expression control element formed by a nucleic acid sequence that permits binding of RNA polymerase and transcription to occur. Promoters are untranslated sequences that are located upstream (5′) to the start codon of a structural gene (generally within about 100 to 1000 bp) and control the transcription and translation of the antigen-specific polynucleotide sequence to which they are operably linked. Promoters may be inducible or constitutive. The activity of the inducible promoters is induced by the presence or absence of biotic or abiotic factors. Inducible promoters can be a useful tool in genetic engineering because the expression of genes to which they are operably linked can be turned on or off at certain stages of development of an organism or in a particular tissue. Inducible promoters can be grouped as chemically-regulated promoters, and physically-regulated promoters. Typical chemically-regulated promoters include, not are not limited to, alcohol-regulated promoters (e.g., alcohol dehydrogenase I (alcA) gene promoter), tetracycline-regulated promoters (e.g., tetracycline-responsive promoter), steroid-regulated promoter (e.g., rat glucocorticoid receptor (GR)-based promoter, human estrogen receptor (ER)-based promoter, moth ecdysone receptor-based promoter, and the promoters based on the steroid/retinoid/thyroid receptor superfamily), metal-regulated promoters (e.g., metallothionein gene-based promoters), and pathogenesis-related promoters (e.g., Arabidopsis and maize pathogen-related (PR) protein-based promoters). Typical physically-regulated promoters include, but are not limited to, temperature-regulated promoters (e.g., heat shock promoters), and light-regulated promoters (e.g., soybean SSU promoter). Other exemplary promoters will be evident to one of skill in the art, and selectable based on the specific circumstances. Many different promoters are well known in the art, as are methods for operably linking the promoter to the gene to be expressed. Both native promoter sequences and many heterologous promoters may be used to direct expression in the packaging cell and target cell. However, heterologous promoters are contemplated, as they generally permit greater transcription and higher yields of the desired protein as compared to the native promoter.

The promoter may be obtained, for example, from the genomes of viruses such as polyoma virus, fowlpox virus, adenovirus, bovine papilloma virus, avian sarcoma virus, cytomegalovirus, a retrovirus, hepatitis-B virus and Simian Virus 40 (SV40). The promoter may also be, for example, a heterologous mammalian promoter, e.g., the actin promoter or an immunoglobulin promoter, a heat-shock promoter, or the promoter normally associated with the native sequence, provided such promoters are compatible with the target cell. In one embodiment, the promoter is the naturally occurring viral promoter in a viral expression system.

Transcription may be increased by inserting an enhancer sequence into the vector(s). Enhancers are typically cis-acting elements of DNA, usually about 10 to 300 bp in length, that act on a promoter to increase its transcription. Many enhancer sequences are now known from mammalian genes (globin, elastase, albumin, α-fetoprotein, and insulin). An enhancer from a eukaryotic cell virus will be used is particularly contemplated. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the antigen-specific polynucleotide sequence, and may be located at a site 5′ from the promoter.

A vector that encodes a core virus is also known as a “viral vector.” There are a large number of available viral vectors that are suitable for use with the invention, including those identified for human gene therapy applications, such as those described by Pfeifer and Verma (Pfeifer, A. and I. M. Verma, 2001, Annu. Rev. Genomics Hum. Genet. 2:177-211, which is incorporated herein by reference in its entirety). Suitable viral vectors include vectors based on DNA viruses. A DNA viral vector may be used, including, for example adenovirus-based vectors and adeno-associated virus (AAV)-based vectors. Likewise, retroviral-adenoviral vectors also can be used with the methods of the invention.

1. Adenoviral Vectors

In particular embodiments, an adenoviral expression vector is contemplated for the delivery of expression constructs, such as T. cruzi antigens and genetic adjuvants. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear, double-stranded DNA, with a genome ranging from 30 to 35 kb in size (Reddy et al., 1998; Morrison et al., 1997; Chillon et al., 1999). An adenovirus expression vector according to the present disclosure comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a iv sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, and function as origins of replication and are necessary for viral DNA replication. The iv sequence is required for the packaging of the adenoviral genome.

A common approach for generating adenoviruses for use as a gene transfer vector is the deletion of the E1 gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1⁻, replication-deficient virus is then proliferated in a “helper” cell line that provides the E1 polypeptides in trans (e.g., the human embryonic kidney cell line 293). Thus, in the present disclosure it may be convenient to introduce the transforming construct at the position from which the E1-coding sequences have been removed. However, the position of insertion of the construct within the adenovirus sequences is not critical to the disclosure. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. Nos. 5,670,488; 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1997) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al. (1990), describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted E1 region and a relocated protein IX gene, wherein the vector expresses a heterologous, mammalian gene.

Other than the requirement that the adenovirus vector be replication defective, or at least conditionally defective, the nature of the adenovirus vector is not believed to be crucial to the successful practice of the disclosure. The adenovirus may be of any of the 42 different known serotypes and/or subgroups A-F. Adenovirus type 5 of subgroup C is a particular starting material in order to obtain the conditional replication-defective adenovirus vector for use in the present disclosure. This is because adenovirus type 5 is a human adenovirus about which a great deal of biochemical and genetic information is known, and it has historically been used for most constructions employing adenovirus as a vector.

As stated above, the typical vector according to the present disclosure is replication defective and will not have an adenovirus E1 region. Adenovirus growth and manipulation is known to those of skill in the art, and exhibits broad host range in vitro and in vivo (U.S. Pat. Nos. 5,670,488; 5,932,210; 5,824,544). This group of viruses can be obtained in high titers, e.g., 10⁹ to 10″ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation for the use of adenoviruses as gene delivery vectors. For example, adenoviral gene delivery-based gene therapies are being developed for liver diseases (Han et al., 1999), psychiatric diseases (Lesch, 1999), neurological diseases (Smith, 1998; Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998; Dorai et al., 1999), pancreatic, bladder (Irie et al., 1999), head and neck (Blackwell et al., 1999), breast (Stewart et al., 1999), lung (Batra et al., 1999) and ovarian (Vanderkwaak et al., 1999).

2. Adeno-Associated Viral Vectors

Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56° C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third, AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

3. RNA Viruses

RNA viruses, such as retrovirus-derived vectors, e.g., Moloney murine leukemia virus (MLV)-derived vectors, and include more complex retrovirus-derived vectors, e.g., lentivirus-derived vectors. Human Immunodeficiency virus (HIV-1)-derived vectors belong to this category. Other examples include lentivirus vectors derived from HIV-2, feline immunodeficiency virus (Hy), equine infectious anemia virus, simian immunodeficiency virus (SIV) and maedi/visna virus.

The viral vector in particular may comprise one or more genes encoding components of the recombinant virus as well as nucleic acids encoding T. cruzi antigens and a genetic adjuvant. In some embodiments, the viral vector encodes components of the recombinant virus, the T. cruzi antigens Tc24 and TSA1, and a genetic adjuvant, such as a dominant negative SHP1. The viral vector may also comprise genetic elements that facilitate expression of the corresponding Tc24, TSA1, and genetic adjuvant sequences in a target cell, such as promoter and enhancer sequences. In order to prevent replication in the target cell, endogenous viral genes required for replication may be removed and provided separately in the packaging cell line.

Any method known in the art may be used to produce infectious retroviral and/or lentiviral particles whose genome comprises an RNA copy of the viral vector. To this end, the viral vector (along with other vectors encoding at least one of: an m1-α subunit and an m1-β subunit of a TCR that recognizes a peptide antigen from Table 1, and optionally, a suicide gene) may be introduced into a packaging cell line that packages viral genomic RNA based on the viral vector into viral particles.

The packaging cell line provides the viral proteins that are required in trans for the packaging of the viral genomic RNA into viral particles. The packaging cell line may be any cell line that is capable of expressing retroviral proteins. Particular packaging cell lines include 293 (ATCC CCL X), Platinum A, HeLa (ATCC CCL 2), D17 (ATCC CCL 183), MDCK (ATCC CCL 34), BHK (ATCC CCL-10) and Cf2Th (ATCC CRL 1430). The packaging cell line may stably express the necessary viral proteins. Such a packaging cell line is described, for example, in U.S. Pat. No. 6,218,181, which is incorporated herein by reference in its entirety. Alternatively a packaging cell line may be transiently transfected with plasmids comprising nucleic acid that encodes one or more necessary viral proteins, including, but not limited to, gag, pol, rev, and any envelope protein that facilitates transduction of a target cell, along with the viral vectors encoding at least one T. cruzi antigen and a genetic adjuvant.

The viral vector may be used to deliver the genes encoding the T. cruzi antigens and genetic adjuvant may be a modified lentivirus, or may be based on a lentivirus. As lentiviruses are able to infect both dividing and non-dividing cells, it is not necessary for target cells to be dividing (or to stimulate the target cells to divide).

In another alternative, the recombinant virus used to deliver the gene of interest may be a modified gammaretrovirus and the viral vector is based on a gammaretrovirus.

The vector may instead be based on the murine stem cell virus (MSCV; (Hawley, R. G., et al. (1996) Proc. Natl. Acad. Sci. USA 93:10297-10302; Keller, G., et al. (1998) Blood 92:877-887; Hawley, R. G., et al. (1994) Gene Ther. 1:136-138, each of which is incorporated herein by reference in its entirety). The MSCV vector provides long-term stable expression in target cells, particularly hematopoietic precursor cells and their differentiated progeny.

The vector may alternatively be based on a modified Moloney virus, for example a Moloney Murine Leukemia Virus. The viral vector can also can be based on a hybrid virus such as that described in Choi, J. K., et al. (2001. Stem Cells 19, No. 3, 236-246, which is incorporated herein by reference in its entirety).

Other vectors also can be used for polynucleotide delivery including vectors derived from herpes simplex viruses (HSVs), including amplicon vectors, replication-defective HSV and attenuated HSV (Krisky D M, Marconi P C, Oligino T J, Rouse R J, Fink D J, et al. 1998. Development of herpes simplex virus replication-defective multigene vectors for combination gene therapy applications. Gene Ther. 5: 1517-30, which is incorporated herein by reference in its entirety).

Other vectors that have recently been developed for gene therapy uses can also be used with the methods of the invention. Such vectors include those derived from baculoviruses and alpha-viruses. Jolly D J. 1999. Emerging viral vectors. pp 209-40 in Friedmann T, ed. 1999. The development of human gene therapy. New York: Cold Spring Harbor Lab, which is incorporated herein by reference in its entirety.

The viral vector construct may comprise sequences from a lentivirus genome, such as the HIV genome or the SIV genome. The viral construct may comprise sequences from the 5′ and 3′ LTRs of a lentivirus. More particularly, the viral construct comprises the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. In particular, the LTR sequences are HIV LTR sequences.

The viral construct may comprise an inactivated or self-inactivating 3′ LTR. The 3′ LTR may be made self-inactivating by any method known in the art. In a particular embodiment the U3 element of the 3′ LTR contains a deletion of its enhancer sequence, such as the TATA box, Spl and NF-kappa B sites. As a result of the self-inactivating 3′ LTR, the provirus that is integrated into the host cell genome will comprise an inactivated 5′ LTR.

Optionally, the U3 sequence from the lentiviral 5′ LTR may be replaced with a promoter sequence in the viral construct. This may increase the titer of virus recovered from the packaging cell line. An enhancer sequence may also be included. Any enhancer/promoter combination that increases expression of the viral RNA genome in the packaging cell line may be used. In a particular embodiment the CMV enhancer/promoter sequence is used.

In some embodiments, the viral construct may comprise an inactivated or self-inactivating 3′ LTR. The 3′ LTR may be made self-inactivating by any method known in the art. In a particular embodiment, the U3 element of the 3′ LTR contains a deletion of its enhancer sequence, such as the TATA box, Spl and NF-kappa B sites. As a result of the self-inactivating 3′ LTR, the provirus that is integrated into the host cell genome will comprise an inactivated 5′ LTR.

The viral construct generally comprises at least one gene encoding a T. cruzi antigen and a gene encoding a genetic adjuvant, such as a dominant negative SHP1. In a viral vector comprising the 5′ LTR and 3′ LTR sequences of a lentivirus, these genes may be located between the 5′ LTR and 3′ LTR sequences. Further, these genes may in particular be in a functional relationship with other genetic elements, for example transcription regulatory sequences such as promoters and/or enhancers, to regulate expression of these genes in a particular manner once the gene is incorporated into the target cell. In certain embodiments, the useful transcriptional regulatory sequences are those that are highly regulated with respect to activity, both temporally and spatially.

In some embodiments, the gene of interest is in a functional relationship with a promoter or enhancer regulatory sequences, such as being operably linked to the genes encoding the T. cruzi antigens and genetic adjuvant.

The promoter or enhancer may be any promoter, enhancer or promoter/enhancer combination known to increase expression of a gene with which it is in a functional relationship. A “functional relationship” and “operably linked” mean, without limitation, that the gene is in the correct location and orientation with respect to the promoter and/or enhancer that expression of the gene will be affected when the promoter and/or enhancer is contacted with the appropriate molecules.

The internal promoter/enhancer may be selected based on the desired expression pattern of the gene of interest and the specific properties of known promoters/enhancers. Thus, the internal promoter may be a constitutive promoter. Non-limiting examples of constitutive promoters that may be used include the promoter for ubiquitin, CMV (Karasuyama et al., 1989. J. Exp. Med. 169:13, which is incorporated herein by reference in its entirety), beta-actin (Gunning et al., 1989. Proc. Natl. Acad. Sci. USA 84:4831-4835, which is incorporated herein by reference in its entirety) and pgk (see, for example, Adra et al., 1987. Gene 60:65-74; Singer-Sam et al., 1984. Gene 32:409-417; and Dobson et al., 1982. Nucleic Acids Res. 10:2635-2637, each of the foregoing which is incorporated herein by reference in its entirety).

In addition, promoters may be selected to allow for inducible expression of the gene. A number of systems for inducible expression are known in the art, including the tetracycline responsive system and the lac operator-repressor system. It is also contemplated that a combination of promoters may be used to obtain the desired expression of the gene of interest. The skilled artisan will be able to select a promoter based on the desired expression pattern of the gene in the organism and/or the target cell of interest.

C. T. cruzi Antigens

T. cruzi antigen compositions can comprise T. cruzi cell lysate, including T. cruzi isolated from an infected patient or animal, or T. cruzi cultured in vitro, or at least one recombinantly expressed T. cruzi protein or peptide. The T. cruzi antigen composition can be a full lysate or a lysate that has been purified or processed as is well known in the art. In certain aspects the T. cruzi antigen is a fully or partially purified recombinant protein(s) or peptide(s). These compositions may increase the prevalence of certain known T. cruzi antigens or other proteins or peptides that enhance the effectiveness of the methods and compositions described herein.

Of particular interest are TSA1 (Trypomastigote surface antigen-1) and Tc24, a flagellar calcium binding protein of 24 kDA. Both of these antigens are present on the surface of T Cruzi, and prophylactic and immunotherapeutic immunization with DNA vaccines encoding these antigens can decrease cardiac tissue damage and amastigote nest density in animal models (Dumonteil et al., 2012; Sanchez-Burgos et al., 2007; Quijano-Hernandez et al., 2008; Limon-Flores et al., 2010).

D. Loading

Dendritic cells can be loaded under conditions and amounts of a T. cruzi antigen composition that is needed to load the MHC of a dendritic cell. See, e.g., U.S. Pat. No. 8,728,806, incorporated herein by reference, in its entirety. As used herein, the term “suitable” for antigen loading are those conditions that permit a DC to contact, process and present one or more T. cruzi antigens on the MHC, whether intracellular or on the cell surface. Based on the present disclosure and the examples herein, the skilled artisan will know the incubation, temperature and time period sufficient to allow effective binding, processing and loading. Incubation steps are typically from between about 1 to 2 to 4 hours, at temperatures of between about 25° to 37° C. (or higher) and/or may be overnight at about 4° C. and the like.

E. Culture

Activation of dendritic cells initiates the process that converts immature DCs, which are phenotypically similar to skin Langerhans cells, to mature, antigen presenting cells that can migrate to the lymph nodes. This process results in the gradual and progressive loss of the powerful antigen uptake capacity that characterizes the immature dendritic cell, and in the up-regulation of expression of co-stimulatory cell surface molecules and various cytokines. Various stimuli can initiate the maturation of DCs. One other consequence of maturation is a change in the in vivo migratory properties of the cells. For example, maturation induces several chemokine receptors, including CCR7, which direct the cells to the T cell regions of draining lymph nodes, where the mature DCs activate T cells against the antigens presented on the DC surface in the context of class I and class II MHC molecules. The terms “activation” and “maturation”, and “activated” and “mature” describe the process of inducing and completing the transition from an immature DC (partially characterized by the ability to take up antigen) to a mature DC (partially characterized by the ability to effectively stimulate de novo T cell responses). The terms typically are used interchangeably in the art.

Known maturation protocols are based on the in vivo environment that DCs are believed to encounter during or after exposure to antigens. The best example of this approach is the use of monocyte conditioned media (MCM) as a cell culture medium. MCM is generated in vitro by culturing monocytes and used as a source of maturation factors, See, US 2002/0160430, incorporated herein by reference. The major components in MCM responsible for maturation are reported to be the (pro)inflammatory cytokines Interleukin 1 beta OL-1β), Interleukin 6 (IL-6) and tumor necrosis factor alpha (TNFα).

Maturation of DCs therefore can be triggered by a multitude of different factors that act via a host of signal transduction pathways. Consequently, there is no single maturation pathway or outcome, but there exists in fact a universe of mature DC stages, each with their own distinct functional characteristics. Conceptually this makes sense because the various threats to the body that the immune system must respond to are manifold, requiring different attack strategies. As an example, while bacterial infection is best cleared by activated macrophages supplemented with specific antibodies, a viral infection is best attacked through cytotoxic T cells that effectively kill virus-infected cells. The killing of cancer cells typically involves a combination of cytotoxic T cells, natural killer cells, and antibodies.

In vitro maturation of DCs can therefore be designed to induce the immune system to favor one type of immune response over another, i.e., to polarize the immune response. Directional maturation of DCs describes the notion that the outcome of the maturation process dictates the type of ensuing immune response that results from treatment with the matured DCs. In its simplest form, directional maturation results in a DC population that produces cytokines that direct a T cell response polarized to either a Tₕ1-type or Tₕ2-type response. DCs express up to nine different Toll-like receptors (TLR1 through TLR9), each of which can be used to trigger maturation. Addition of interferon gamma (IFN-γ) to most maturation protocols results in the production of interleukin 12 by the mature DCs, which dictates a Tₕ1-type response. Conversely, inclusion of prostaglandin E2 has the opposite effect.

Factors that can be used in the directional maturation of activated DCs can therefore include for example, Interleukin 1 beta (IL-β), Interleukin 6 (IL-6), and tumor necrosis factor alpha (TNFα). Other maturation factors include prostaglandin E2 (PGE2), poly-dIdC, vasointestinal peptide (VIP), bacterial lipopolysaccharide (LPS), as well as mycobacteria or components of mycobacteria, such as specific cell wall constituents. Additional maturation factors include for example, an imidazoquinoline compound, e.g., R848 (WO 00/47719, incorporated herein by reference in its entirety), a synthetic double stranded polyribonucleotide, agonists of a Toll-like receptor (TLR), such as TLR3, TLR4, TLR7 and/or TLR9, a sequence of nucleic acids containing unmethylated CpG motifs known to induce the maturation of DC, and the like. Further, a combination of any of the above agents can be used in inducing the maturation of dendritic precursor cells.

Fully mature dendritic cells differ qualitatively and quantitatively from immature DCs. Once fully mature, DCs express higher levels of MHC class I and class II antigens, and higher levels of T cell costimulatory molecules, i.e., CD80 and CD86. These changes increase the capacity of the dendritic cells to activate T cells because they increase antigen density on the cell surface, as well as the magnitude of the T cell activation signal through the counterparts of the costimulatory molecules on the T cells, e.g., CD28 and the like. In addition, mature DCs produce large amounts of cytokines, which stimulate and polarize the T cell response.

Generally methods for ex vivo DC generation comprise obtaining a cell population enriched for DC precursor cells from a patient and then differentiating the DC precursor cells in vitro into mature DCs prior to introduction back into the patient. Typically, to generate immature dendritic cells (DC), one must first purify or enrich the monocytic precursors from other contaminating cell types. This is commonly done through adherence of the monocytic precursors to a plastic (polystyrene) surface, as the monocytes have a greater tendency to stick to plastic than other cells found in, for example, peripheral blood, such as lymphocytes and natural killer (NK) cells. After substantially removing the contaminating cells by vigorous washing, the monocytes are cultured with cytokines that convert the monocytic precursors to either immature DC or directly to mature DC. Methods for differentiating the monocytic precursor cells to immature DC were first described by Sallusto and Lanzavecchia (J. Exp. Med., 179:1109-1118, 1994, incorporated herein by reference), who used the cytokines GM-CSF and IL-4 to induce the differentiation of the monocytes to immature DC. While this combination of cytokines is most typically used, various other combinations have been described to accomplish the same goals, such as replacing IL-4 with IL-13 or IL-15. The end result of this process is a “veiled” cell, which expresses T cell costimulatory molecules, as well as high levels of molecules of the major histocompatibility complex (MHC), but does not express the dendritic cell maturation marker CD83. These cells are similar to Langerhans cells in the skin, and their prime physiological function is to capture invading microorganisms.

Variations on this method include different methods of purifying monocytes, including, for example, tangential flow filtration (TFF), or by binding antibodies attached to beads to surface molecules on the monocytes. The beads with the bound cells are then concentrated in a column, or on a magnetic surface, such that contaminating cells can be washed away, after which the monocytes are eluted off the beads. In yet another method to obtain dendritic cells precursors, cells expressing the stem cell marker CD34, either from blood (U.S. Pat. No. 5,994,126, incorporated herein by reference) or from the bone marrow are purified. These cells can be cultured with the essential cytokine GM-C SF to differentiate into immature DC. These DC apparently have very similar characteristics and functional properties as immature DC generated from monocytes.

Immature DC have a high capacity for taking up and processing antigens, but have a limited ability to initiate immune responses. The ability to initiate an immune response is acquired by maturation of the immature DC. This maturation is also referred to as activating, or activation of, the DC. The maturation process is initiated through contact with maturation-inducing cytokines, tumor antigen compositions and/or nucleic acid compositions, and the like, as described herein.

### IV. Therapeutic Methods and Compositions

A. Pharmaceutical Compositions

In certain embodiments, the present invention concerns formulation of one or more dendritic cell compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized.

In certain aspects of the present invention, pharmaceutical compositions are provided comprising one or more of the dendritic cell compositions described herein in combination with a pharmaceutically acceptable carrier. In certain preferred embodiments, the pharmaceutical compositions of the invention comprise immunologic cells of the invention for use in prophylactic and therapeutic applications. Generally, such compositions will comprise one or more dendritic cell composition of the present invention, optionally in combination with one or more immunostimulants and/or T. cruzi treatments.

B. Administration of Cell Populations

In another aspect of the invention, methods are provided for administration of mature dendritic cells, or a cell population containing such cells, to a subject in need thereof. In certain embodiments, such methods are performed by obtaining dendritic cell precursors or immature dendritic cells, differentiating and maturing those cells in the presence of a nucleic acid composition and a tumor antigen composition to form a mature dendritic cell population primed towards Th-1 response. The immature dendritic cells can be contacted with antigen prior to or during maturation. Such mature, primed dendritic cells can be administered directly to a subject in need of immunostimulation.

In a related embodiment, the mature dendritic cells can be contacted with lymphocytes from a subject to stimulate T cells within the lymphocyte population. The activated, polarized lymphocytes, optionally followed by clonal expansion in cell culture of antigen-reactive CD4+ and/or CD8+ T cells, can be administered to a subject in need of immunostimulation. In certain embodiments, activated, polarized T cells are autologous to the subject.

In another embodiment, the dendritic cells and the recipient subject have the same MHC (HLA) haplotype. Methods of determining the HLA haplotype of a subject are known in the art. In a related embodiment, the dendritic cells are allogenic to the recipient subject. For example, the dendritic cells can be allogenic to the recipient which has the same MHC (HLA) haplotype. The allogenic cells are typically matched for at least one MHC allele (e.g., sharing at least one but not all MHC alleles).

### V. Adoptive Immunotherapy Methods

As used herein, treating a subject using the compositions and methods of the present invention refers to reducing the symptoms of the disease, reducing the occurrence of the disease, and/or reducing the severity of the disease. Treating a subject can refer to the ability of a therapeutic composition of the present invention, when administered to a subject, to prevent a disease from occurring and/or to cure or to alleviate disease symptoms, signs or causes. As such, to treat a subject means both preventing disease occurrence (prophylactic treatment) and treating a subject that has a disease (therapeutic treatment). In particular, treating a subject is accomplished by providing or enhancing an immune response in the subject.

More specifically, therapeutic compositions as described herein, when administered to a subject by the methods of the present invention, preferably produce a result which can include alleviation of the disease, elimination of the disease, reduction of symptoms associated with the disease, elimination of symptoms associated with the disease, prevention of a secondary disease resulting from the occurrence of a primary disease, and prevention of the disease.

In certain embodiments, in vitro or in vivo generated T_{H}1-polarized CD8+DCs are used as adoptive immunotherapy for amelioration of disease symptoms.

In some of these embodiments, the T_{H}1-polarized CD8+DCs are autologous/syngeneic to the subject and present antigen(s) associated with the aberrant immune response. For example, immature DCs can be harvested from a subject and treated in vitro with a T_{H}1-polarizing composition that contains the antigen(s) of interest (e.g., T. cruzi antigens). The resultant mature DC can then be administered to the subject. In some embodiments, a single antigen or antigenic peptide is included in the tumor antigen composition whereas in other embodiments, more than one antigen or antigenic peptide may be used, including 2, 3, 4, 10 or more including a cell lysate or varying purities. Additionally, multiple independently generated DCs can be administered to a subject. Furthermore, administration of DCs to a subject can be done as often as is required to ameliorate the symptoms associated with the disease state.

In other of these embodiments, the DCs are allogeneic to the subject. For example, immature dendritic cells can be harvested from an organ donor and treated in vitro with a nucleic acid and tumor antigen composition. The resultant allogeneic mature DCs can then be administered to the subject to promote the cure or treatment of disease in that subject.

In certain embodiments, in vivo or in vitro generated cells are used in an adoptive immunotherapy method to ameliorate symptoms associated with a disease, such as Chagas disease, in a subject.

In certain embodiments of the adoptive immunotherapy methods described above, the cells of interest (i.e., mature DCs) can be purified prior to administration to the subject. Purification of the cells can be done using a variety of methods known in the art, including methods in which antibodies to specific cell surface molecules are employed. These methods include both positive and negative selection methods. For example, cells generated in vitro can be isolated by staining the cells with fluorescently labeled antibodies to cell surface markers followed by sorting of the cells that express both of these markers on their cell surface using fluorescence activated cell sorting (FACS). These and other purification/isolation methods are well known to those of skill in the art.

The mature DCs of the invention either can be used immediately after their generation (and purification, if applicable) or stored frozen for future use. In certain embodiments, enough mature DCs are generated to provide an initial dose for the subject as well as cells that can be frozen and stored for future use if necessary.

In certain other embodiments, mature DCs can be expanded in vitro from freshly isolated or frozen cell stocks to generate sufficient numbers of cells for effective adoptive immunotherapy. By effective dose it is meant enough cells to ameliorate at least one symptom caused by the disease of interest, such as Chagas Disease associated chronic cardiomyopathy. The methods for determination of an effective dose for therapeutic purposes is known in the art. The expansion of the cells can be achieved by any means that maintains their functional characteristics. The phenotypic and functional properties of the resultant expanded cells can be tested prior to their therapeutic use and/or storage to verify that the expansion process has altered their activity.

### VI. Expression Assays

One application of interest is the examination of gene expression in mature DCs of the invention. The expressed set of genes may be compared with a variety of cells of interest, e.g. other DCs, etc., as known in the art. For example, one could perform experiments to determine the genes that are regulated during development of the maturation processes.

Any suitable qualitative or quantitative methods known in the art for detecting specific mRNAs can be used. mRNA can be detected by, for example, hybridization to a microarray, in situ hybridization in tissue sections, by reverse transcriptase-PCR, Northern blots containing poly(A) mRNA, or next gen. sequencing methods. One of skill in the art can readily use these methods to determine differences in the size or amount of mRNA transcripts between two samples. For example, the level of particular mRNAs in DCs is compared with the expression of the mRNAs in a reference sample.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of polynucleotides, particularly polynucleotides corresponding to one or more differentially expressed genes.

Alternatively, gene expression in a test sample can be performed using serial analysis of gene expression (SAGE) methodology (Velculescu et al., Science (1995) 270:484). SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific polynucleotide sequences (or restriction enzyme sites) are used as unique identifiers of genes, coupled with information about fragment length or fragment location within the expressed gene. The relative representation of an expressed gene with in a sample can then be estimated based on the relative representation of the fragment associated with that gene within the pool of all possible fragments. Methods and compositions for carrying out DD are well known in the art, e.g., U.S. Pat. Nos. 5,776,683; and 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry).

Methods for collection of data from hybridization of samples with arrays are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639.

In another screening method, the test sample is assayed at the protein level. Analysis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry, microscopy, radiography, scintillation counting, etc. Any suitable alternative methods of qualitative or quantitative detection of levels or amounts of differentially expressed polypeptide can be used, for example ELISA, western blot, immunoprecipitation, radioimmunoassay, etc.

### VII. Screening Assays

The subject cells are useful for in vitro assays and screening to detect or characterize cells contributing to a disease state. A wide variety of assays may be used for this purpose, including immunoassays for protein binding; determination of cell growth, differentiation and functional activity; production of cytokines; and the like.

In screening assays for biologically active agents the subject cells, usually a culture or a biopsy comprising the subject cells, is contacted with the agent of interest, and the effect of the agent assessed by monitoring output parameters, such as expression of markers, cell viability, and the like. The cells may be freshly isolated, cultured, genetically altered, or the like.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc., or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

### VIII. Kits

The present invention further pertains to a packaged pharmaceutical composition for producing Tₕ1 dendritic cells such as a kit or other container. The kit or container holds an effective amount of a pharmaceutical composition for carrying out the methods or producing the compositions described herein and/or instructions for producing or using the compositions for therapy of a patient or subject having or suspected of having or at risk of developing a T. cruzi infection. The pharmaceutical composition includes at least one nucleic acid, polypeptide, or antibody of the present invention, in an effective amount such that the selected cancer is controlled. The kit may also contain various reagents and containers for monitoring the isolation and maturation and function of Tₕ1 dendritic cells.

### IX. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Construction of adenoviral vaccine vector. The construction of the replication-deficient human Type 5 recombinant adenovirus vector carrying genetic adjuvants caAKT (constitutively-activated Akt) (Sun et al., 2010), iMC (inducible MyD88/CD40) (Narayanan et al., 2011), or dnSHP (dominant negative SHP-1 (Zhang et al., 2000) upstream of the T. cruzi antigens Tc24 and TSA1 was performed as described previously (Kemnade et al., 2012). Cells were loaded with recombinant Tc24 protein (SEQ ID NO: 1) and transduced with an adenoviral vector comprising genes encoding a dominant negative kinase-inactivated SHP1 protein (SEQ ID NO: 4 encodes the dnSHP protein with amino acid sequence SEQ ID NO: 3) as well as the Tc24 (SEQ ID NO: 2 encodes the Tc24 protein with amino acid sequence SEQ ID NO: 1) and TSA1 (SEQ ID NO: 6 encodes the TSA1 protein with amino acid sequence SEQ ID NO: 5).

Briefly, HA-tagged genetic adjuvants (Akt, iMC and dnSHP (SEQ ID NO: 4, encoding the dnSHP protein of SEQ ID NO: 3)), generated as described previously, were amplified by PCR using XbaI-flanked primers and cloned into the XbaI site of the pShuttle plasmid (Ramachandran et al., 2011; Sun et al., 2010; Narayanan et al., 2011). The T. cruzi antigens Tc24 (SEQ ID NO: 2 encoding the Tc24 protein of SEQ ID NO: 1) and TSA1 (SEQ ID NO: 6, encoding the TSA1 protein of SEQ ID NO: 5) were cloned as a PCR-generated fusion fragment downstream of the adjuvants between NheI and NotI sites. A single primer-encoded glycine hexamer linker separated the antigens while a primer-encoded P2A sequence inserted between antigens and adjuvant permitted cleavage of the two (Kim et al., 2011). The expression cassette was digested and cloned into the PmeI site of the pShuttle plasmid. The cassette was excised using the unique restriction sites Psce-I and Iceu-I and ligated into the Adeno-X backbone. Using the BD-Adeno-X system, low titer adenovirus was made by transfecting HEK293 cells with the pAdenoX construct expressing the T. cruzi specific antigens according to the manufacturer's instructions (BD Biosciences, San Jose, Calif.).

Western Blotting and Analysis. All gel electrophoreses were performed under denaturing, reducing conditions on a 12% polyacrylamide gel with subsequent transfer to a 0.45 μm nitrocellulose membrane for antibody probing. All blocking and antibody staining steps were carried out in 5% milk, and primary antibodies were applied overnight at 4° C. Western blot chemiluminescent signal was detected using a ChemiDoc XRS digital imaging system supported by Image Lab software Version 2.0.1 (Bio-Rad Laboratories, Hercules, Calif.). All Western blots were quantitated by densitometry of Ponceau S (Sigma-Aldrich, St. Louis, Mo.) stained membranes. Contamination of supernatants with residual cell lysate or debris from cell death was controlled for by immunoblotting with anti-β-actin (Santa Cruz Biotechnology, Dallas, Tex.) and additional densitometry. Densitometry was performed using ImageJ software (NIH; Bethesda, Md.). All western blots are representative of at least three independent experiments.

Vaccine production. DC were generated from Balb/c mice. Bone marrow leukocytes were flushed from mouse tibia and femur and cultured in Aim-V containing 10% FBS and 1% antibiotics and supplemented with 50 ng/mL mGM-CSF and 10 ng/mL mIL-4 for three days. Cells were cultured in a humidified chamber at 37° C. and 5% atmospheric CO₂. The culture medium was removed and replenished with an equal volume of fresh medium supplemented with cytokines on day three. On day five, the cells were replenished with fresh AIM-V containing 10% mouse serum and 1% penicillin/streptomycin/amphotericin (anti-anti) and supplemented with mGM-CSF and mIL-4 (R&D Systems, Minneapolis, Minn.). The immature DC were harvested on day six, counted, and incubated with adenovirus (expressing Tc24 (SEQ ID NO: 2) and TSA1 (SEQ ID NO: 6) antigens and dnSHP (SEQ ID NO: 4) as genetic adjuvant) and recombinant Tc24 protein (SEQ ID NO: 1) (10 ug/ml) in AIM-V supplemented with 5% mouse serum for three hours (Martinez-Campos et al., 2015). The virus was added to the cells at a concentration of 1,000 viral particles per DC. Cells were plated in a six-well plate at 10⁶ cells/well in AIM-V supplemented with 5% Mouse Serum. After three hours of incubation, DC were matured for 24 hours in AIM-V supplemented with 10% mouse-serum, 50 ng/ml GM-CSF, 10 ng/ml IL-4, 10 ng/ml IL-1β (R&D Systems), 10 ng/ml TNF-α (R&D Systems), 15 ng/ml IL-6 (R&D Systems), and 1 μg/ml PGE₂ (Sigma-Aldrich). The final DC product was extensively characterized as described previously (Konduri et al., 2013; Konduri et al., 2016).

Mice, parasites, and infection model. Six to eight-week-old female Balb/c mice were obtained from Harlan Laboratories (Houston, Tex.). All mice were maintained in accordance with the specific IACUC requirements of Baylor College of Medicine (Animal Welfare Assurance Number: A-3823-01) and in specific accordance with IACUC-approved protocol AN-5973. T. cruzi H1 (Tc I) strain parasites, previously isolated from a human case in Yucatan, Mexico, were maintained by serial passage in mice. Naïve mice were infected intraperitoneally with 500 blood form trypomastigotes as previously described and validated (Dumonteil et al., 2004). Seven days post-infection (dpi) the mice were immunized intraperitoneally with 250,000 DC loaded with adenoviral particles and Tc24 protein. Blood was collected twice weekly for quantification of parasitemia. At 50 dpi, all remaining mice were sacrificed and analyzed.

Vaccination, blood draws and parasitemia. Seven days post parasite challenge, cohorts of 4-5 mice each received primary vaccination with 250,000 DC per mouse intraperitoneally. Boost vaccination was given seven days later (Konduri et al., 2013; Konduri et al., 2016). Blood was collected from tail vein twice per week, and parasitemia was quantitated by visual microscopy and qPCR using primers specific for T. cruzi nuclear DNA (Melo et al., 2015). Serum was collected at every blood draw for downstream analysis. Heart tissue was collected on the day of sacrifice for histopathology and qPCR analysis. Experiment characterized is one of three different iterations.

IFN-γ ELISPOT assays. Tc24-specific IFN-γ-producing splenocytes were quantified by ELISpot after overnight bulk restimulation with Tc24-loaded DC using the IFN-γ ELISpot PLUS kit (Mabtech, Inc, Cincinnatti, Ohio) according to the manufacturer's instructions. Briefly, filter plates were coated overnight with 15 μg/ml coating antibody. Plates were blocked with DMEM supplemented with 10% FBS at room temperature. Cells and stimuli were added to the plate in a final volume of 0.2 ml. Cells were used at final concentrations of 2.5×10⁵ cells per well, 5 μg/ml concanavalin A, 10 μg/ml Tc24 protein (SEQ ID NO: 1), or media only (DMEM supplemented with 5% FBS, 1× Pen/Strep). The plate was incubated for approximately 18 hours at 37° C., 5% CO₂. Bound IFN-γ was quantified by 1 μg/ml biotinylated detection antibody, 1:1,000 dilution of Streptavidin HRP and TMB substrate. After drying a minimum of 18 hours, spots were counted manually using a dissecting microscope.

Serum antibody titers. Serum antibodies to Tc24 were measured by ELISA. Plates were coated with 1.25 μg/ml Tc24 protein in coating solution. Plates were blocked and serially diluted serum samples were added. Bound antibody was detected with 1:4,000 HRP-conjugated goat anti-mouse total IgG, IgG₁ or IgG₂ₐ secondary antibody. The reaction was developed with TMB Substrate (Thermo Fisher Scientific, Waltham, Mass.). Titers were recorded as the last positive dilution above a cut-off OD as determined by the OD₄₅₀+3 SD of serum from naïve mice.

Evaluation of parasite burdens. Total DNA was isolated from blood or tissue using a DNEasy blood and tissue kit (Qiagen, Valencia, Calif.). T. cruzi levels were measured from 10 ng blood DNA or 50 ng heart tissue DNA using quantitative real-time PCR using Taq-Man® Fast Advanced Master Mix (Life Technologies, Carlsbad, Calif.) and oligonucleotides specific for the satellite region of T. cruzi nuclear DNA (primers 5′ AATCGGCTGATCGTTTTCGA 3′ (SEQ ID NO: 7) and 5′ AATTCCTCCAAGCAGCGGATA 3′ (SEQ ID NO: 8), probe 5′ 6-FAM CACACACTGGACACCAA MGB 3′ (SEQ ID NO: 9), Life Technologies, 4304972, 4316032) (Konduri et al., 2016; Melo et al., 2015). Data were normalized to GAPDH (primers 5′ CAATGTGTCCGTCGTGGATCT 3′ (SEQ ID NO: 10) and 5′ GTCCTCAGTGTAGCCCAAGATG 3′ (SEQ ID NO: 11), probe 5′ 6-FAM CGTGCCGCCTGGAGAAACCTGCC MGB 3′ (SEQ ID NO: 12), Life Technologies) (Piron et al., 2007), and parasite equivalents were calculated based on a standard curve as described previously (Gangisetty and Reddy, 2009; Caldas et al., 2012).

Evaluation of cardiac pathology. Heart tissue was removed from euthanized animals and fixed in 10% formaldehyde for histopathological analysis. Samples were embedded in paraffin, cut into 5 μM sections, and stained with hematoxylin and eosin. For each mouse, a representative section was identified and amastigote nests were quantified over 20 fields of view at 400× magnification in a blinded fashion. Lymphocytic infiltration in the representative sections was scored on a scale of 0 to 5, with 0 being minimum or no infiltration and 5 being maximum infiltration (Gupta and Garg, 2013). Heart enlargement was determined by measurement of multiple cross-sectional slices (>3 for each animal) taken from the center of each paraffin block.

Quantification of Cardiac Fibrosis. Heart samples were fixed in 10% neutral buffered formalin and embedded in paraffin. To measure cardiac fibrosis, 5 μm sections were adhered to glass slides and stained with Masson's Trichrome stain. Images of three to five representative sections from each mouse were captured at 100× magnification using a Fisher Micromaster microscope and Micron software. Images were evaluated by a reviewer blinded to the treatment groups and analyzed using ImageJ FIJI software to quantify the area of fibrosis and total tissue area.

Statistical analysis. Statistical significance was determined by Student's two-tailed t test or one-way ANOVA using Prism Software (GraphPad Software, La Jolla, Calif.). Survival was analyzed by means of Kaplan-Meier analysis. Bonferroni correction was applied when necessary to control for type I errors during multiple comparisons. Data are presented as the mean±SEM unless stated otherwise. Statistical significance was defined as p<0.05.

### Example 2—Results and Discussion

The objective of the present study was to determine the therapeutic efficacy of dendritic cell-based vaccines to prevent progression of acute Chagas disease to Chagasic cardiomyopathy. Vaccines using replication-deficient human recombinant type 5 adenoviruses have been viewed an attractive strategy to deliver antigens for the generation of specific immune responses, and it has previously been shown that the administration of DNA vaccines encoding a trans-sialidase antigen (TSA1) or the secreted antigen Tc24 following a T. cruzi infection can reduce parasitemia and cardiac influiammatory reactions while increasing survival of treated mice (Kim et al., 2011; Cencig et al., 2011). Therapeutic DNA vaccination with plasmids encoding Tc24 and TSA1 antigens demonstrated efficacy across different strains of mice with no antigenic interference and/or genetic restriction of the vaccine efficacy (Limon-Flores et al., 2010). As in mice, the therapeutic administration of two doses of DNA vaccines encoding TSA1 and Tc24 during the acute phase has also been tested in dogs and EKG recording indicated a decrease in the severity of disease-associated cardiac arrhythmia (Quijano-Hernandez et al., 2008). Further, genetic adjuvantation provides yet another attractive strategy by which to augment the generation of immune responses (Hanks et al., 2005), and previous work has shown that dendritic cells transduced with a dominant negative kinase-inactivated SHP-1 adjuvant (dnSHP) can substantially enhance T-cell responses (Ramachandran et al., 2011).

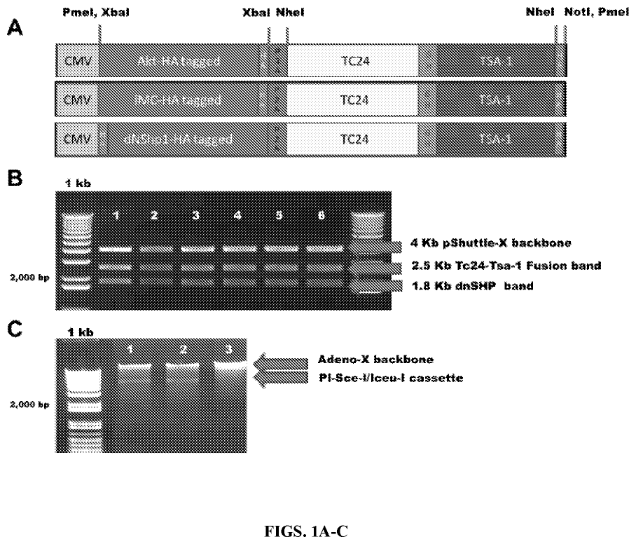

Therefore, bicistronic adenoviral constructs encoding functional adjuvants and parasite specific antigens were constructed for to determine whether these adenoviral constructs would reduce parasitemia and cardiac inflammatory reactions. Shuttle plasmids were constructed with the T. cruzi antigens Tc24 (SEQ ID NO: 2) and TSA1 (SEQ ID NO: 6) downstream of genetic adjuvants Akt (Sun et al., 2010), iMC (Narayanan et al., 2011), and dnSHP (SEQ ID NO: 4) (Ramachandran et al., 2011) (FIG. 1A). Ampicillin resistant positive clones were sequenced followed by restriction digestions with XbaI and PmeI to confirm successful cloning (FIG. 1B). The constructs were excised from the shuttle plasmid backbone and ligated into the adenoviral backbone following the manufacturer's instructions. Ampicillin resistant clones were selected and confirmation of positive clone selection was performed by Psce-I and Iceu-I restriction digest followed by direct sequencing. (FIG. 1C).

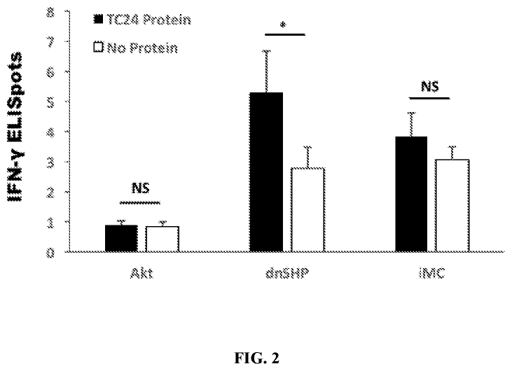

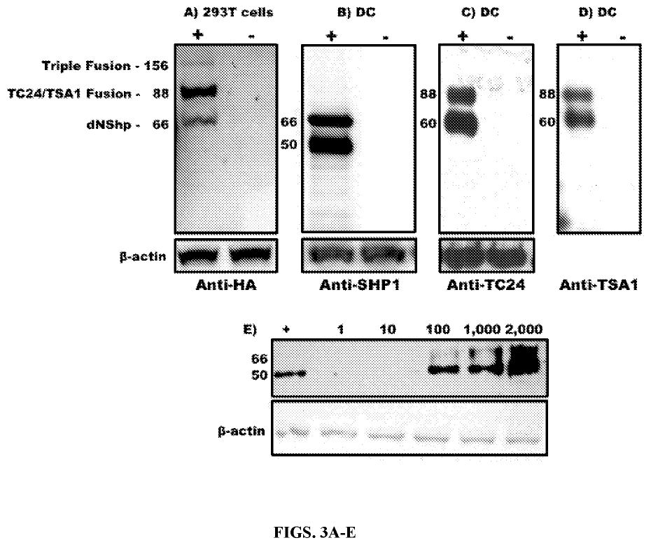

Rodents are a widespread natural reservoir for T. cruzi, and laboratory mice have been used extensively to study vaccine efficacy as well as T. cruzi effects on cardiac pathology induced by both acute and chronic Chagas disease (Dumonteil et al., 2004; Gupta and Garg, 2013). Inbred rodent models also represent the most accurate, cost-effective, and reproducible systems in which to effectively model translational research with statistically reliable results. Therefore, following preparation of the adenovirus constructs, the efficacy of each in generating antigen specific immune responses was tested. Mice were vaccinated with each of the adenoviral constructs (caAKT-Tc24/TSA1, iMC-Tc24/TSA1 and dnSHP-Tc24/TSA1) and antigen specific responses were measured by IFN-γ secretion of restimulated splenocytes. Compared to caAKT and iMC, genetic adjuvantation with dnSHP resulted in significantly increased enhancement of the production of Tc24-specific IFN-γ responses (FIG. 2). Low titer adenovirus was produced for the construct with dnSHP as genetic adjuvant. Western blot analysis of 293T cells and dendritic cells transduced with the viral particles and probed with anti-HA, anti-SHP-1, anti-Tc24 and anti-TSA1 proteins confirmed the expression of the genetic adjuvant and/or antigenic fusion protein (FIGS. 3A-3D).

There exist legitimate concerns about the ability of pre-existing Ad5 neutralizing antibody titers to hinder or abolish in vivo transduction of genetic materials in subjects already exposed, so it was sought to bypass this by transducing dendritic cells (DC) directly with the adenovirus in vitro. To determine whether antigen specific cellular immune responses against Tc24 would be mounted following vaccination with an adenoviral vector-transduced dendritic cell-based vaccine, DC were transduced with different titrations of the viral particles, and cell lysates were probed with anti-SHP1 antibody to determine the optimum titer at which the antigen, adjuvant and fusion proteins could be detected. Previous studies have reported using 10¹⁰ virus particles (vp) per cell for transduction (Sun et al., 2010), however, it was observed that good expression could be obtained by transducing dendritic cells with doses as low as 100 (10²) vp per cell (FIG. 3E).

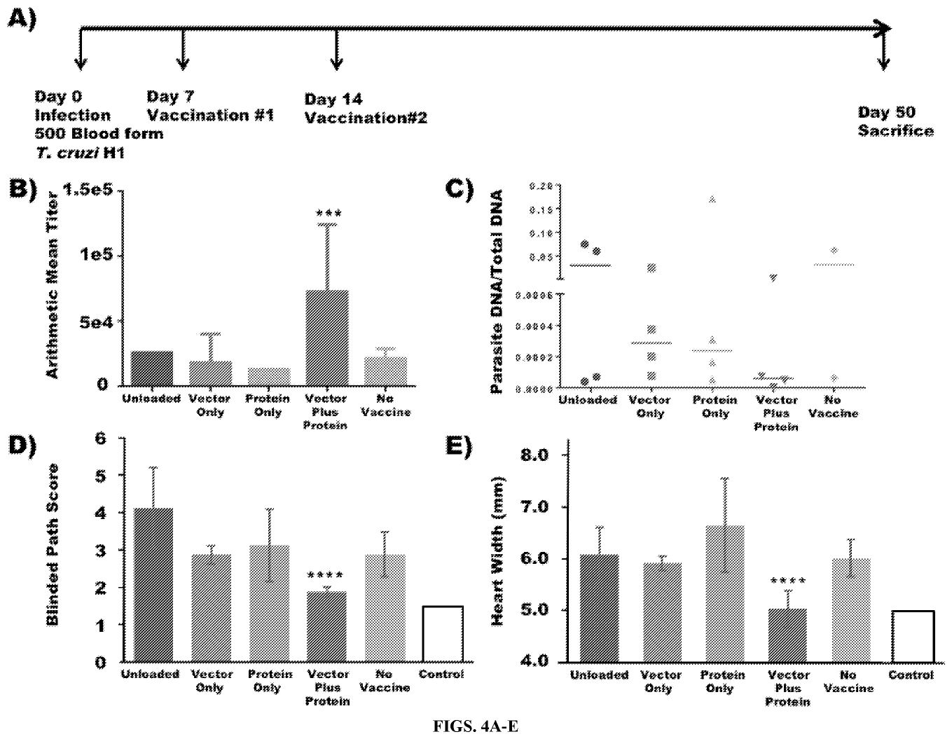

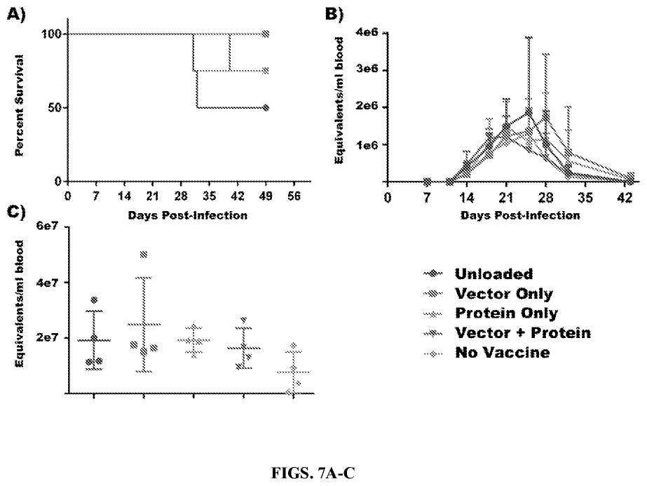

DC vaccines transduced with dnSHP adenoviral construct and loaded with Tc24 recombinant protein reduce T. cruzi parasite burden and significantly improve cardiac pathology. To test the therapeutic efficacy of the dnSHP-based DC vaccine, mice were challenged i.p. with 500 blood form T. cruzi trypomastigotes, and seven days later treated therapeutically using a variety of different DC-based platforms. These included unloaded DC, DC transduced with the dnSHP vector alone, DC loaded with only Tc24 recombinant protein, DC both transduced with the dnSHP vector and simultaneously loaded with Tc24 recombinant protein to provide antigen-specific T-cell help (Konduri et al., 2013; Decker et al., 2006; Decker et al., 2009), as well as untreated controls, as shown in FIG. 4A. Following infection and treatment, mice were monitored for 50 days and then sacrificed for analysis. Most mice lived until day 50, though a few died prior to day 50 and were not available for all analyses. A Kaplan-Meier survival plot is shown in FIG. 7, however there are no statistically significant differences in overall survival observed between groups. Analysis of serum antibody titers demonstrated that mice treated with vector+protein-loaded DC exhibited significantly elevated Tc24-specific IgG₁ antibody titers (p<0.004) (FIG. 4B). Mice treated by any other means exhibited IgG₁ titers indistinguishable from those stimulated by infection alone. There were no statistically significant differences observed among Tc24-specific IgG₂ₐ or IgG_{2b} antibody isotype titers nor significant differences observed in total Tc24-specific serum IgG (not shown). Further, there were no statistically significant differences in IFN-γ-secreting T-cells in peripheral circulation as evidenced by an IFN-γ ELISpot (data not shown).

Despite only modest immunological differences observed between groups on study day 50, there were significant differences observed with regard to objective cardiac pathology. Quantitative PCR analysis of cardiac samples using T. cruzi specific primers indicated a clear trend toward low or no detectable parasite DNA present among mice that received vector+protein loaded DC: 76% less parasite DNA than protein only DC, 80% less than vector only DC, and >99% less than either unloaded DC or unvaccinated (FIG. 4C). However, there were no clear differences in blood form parasite burden between groups (FIG. 7B, and area under the parasitemia curve shown for each animal in FIG. 7C).

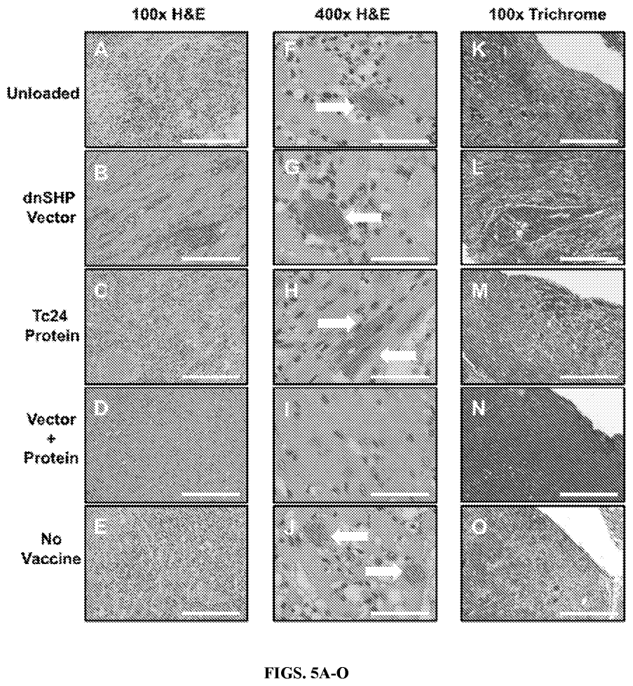

H&E stained cardiac tissue were scored for lymphocytic infiltration in a randomized and blinded fashion. A score of 0 indicated minimum or no infiltration and a score of 5 indicated maximum infiltration. Similar to the qPCR results, the pathological scoring demonstrated that mice which received vector+protein loaded DC exhibited substantially lower pathological index scores, some even on par with those of uninfected mice (p<0.0006) (FIG. 4D). Further, cardiac size, or enlargement, as assessed by multiple measurements of gross cross-sectional distance, was nearly 20% less (5.04 mm vs 6.16 mm, p<0.0001) among mice that received vector+protein loaded DC, and similar to that of uninfected mice (FIG. 4E). Representative 100× cardiac histopathology images of each group are shown in FIGS. 5A-5E, as well as the presence of amastigote nests in the 400× images, as depicted in FIGS. 5F-5J. Significant numbers of amastigote nests were observed in at least one animal of each of the five groups with the exception of the vector+protein loaded DC group, in which no amastigote nests were observed in any sample of any animal. Therefore, vaccination with class I and II presentation of Tc24 and TSA-1 significantly decreased total fibrotic cardiac tissue area and overall heart width.

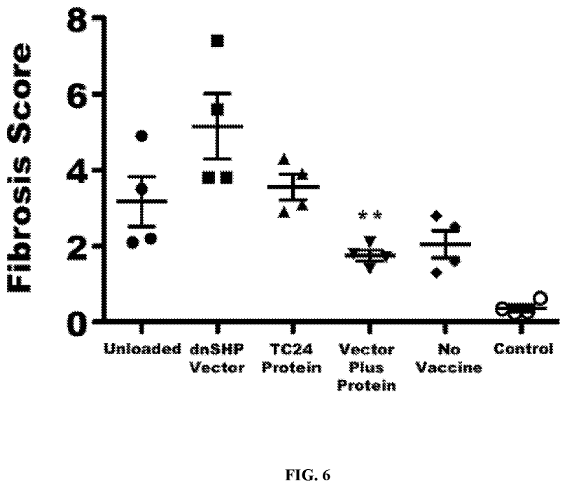

The presence of inflammatory fibrosis was also characterized, using Masson's Trichrome staining with blinded image capture and quantitation in ImageJ. As indicated in the representative images (FIGS. 5K-50), extensive inflammatory damage was observed in all groups except the vector+protein group and, to a lesser extent, the unvaccinated group. Nonetheless among infected animals, trichrome staining was significantly lower only in the vector+protein group (**p<0.01 by one-way ANOVA) (FIG. 6).

This study has demonstrated that expression of Tc24 and TSA1 antigens through dendritic cell-based vaccination generated effective antigen-specific immune responses that not only aided in parasite clearance but also mitigated cardiac manifestations of the parasite. In this model system, vaccination with class I and II presentation of Tc24 and TSA-1 significantly decreased total fibrotic cardiac tissue area and overall heart width. Detailed studies of the Chagasic infection process in vitro have shown that invasion of T. cruzi into cardiac cells induces hypertrophy and increased collagen IV and fibronectin (Garzoni et al., 2008). Further, T. cruzi invasion activates TGF-β, inducing intracellular signaling cascades that can drive expression of pro-fibrotic genes (Udoko et al., 2016; Waghabi et al., 2005; Lin et al., 2005; Shi and Massague, 2003; Neilson, 2005). Thus, it is proposed that vaccine efficacy may be attributed in part to inducing an immune response that enhances parasite killing, decreasing total cardiomyocyte invasion by T. cruzi with concomitant reduction of TGF-β-activated pro-fibrotic gene expression.

Generation of differential IgG subclasses have been indicated in antibody responses (Bryan et al., 2010; el Bouhdidi et al., 1994) as well as CD8 responses after T. cruzi infection (Padilla et al., 2009; Martin and Tarleton, 2004). In the present study, analysis of the IgG subclasses from the mice immunized with adenoviral dendritic cell vaccine indicated IgG₁ being the dominant response in immunized Balb/c mice. It has previously been shown that a strong IgG_{2b} response induced by the Ad5-gp83 vaccine facilitated antibody dependent cellular cytotoxicity (ADCC) and complement-dependent cytotoxicity in addition to eliciting neutralizing antibodies (Farrow et al., 2014). Coordinated cellular (T_{H}1) and humoral (T_{H}2) responses are important for effective clearance of intracellular pathogens by the adaptive immune system. In conjunction with an adjuvant, protective T_{H}2 immunity can be achieved by administration of the antigens. In the case of intracellular infection, protective cell based responses can only be generated by administration of attenuated live pathogen that induces subclinical infection. In the current study, the loading of DC with both adenovirus and recombinant protein together resulted in a significant amelioration of cardiac pathology in comparison to mice that received DC loaded by any other means. While the reasons for this observation are not elucidated here, they may be related to the previously-observed and published phenomenon of homologous T-cell help (Konduri et al., 2016; Decker et al., 2006; Decker et al., 2009).

Interestingly, some of the vaccinated groups in this acute model developed significant cardiac fibrosis. Evident fibrosis takes time to occur and, with only seven weeks between inoculation and sacrifice, significant cardiac fibrosis was an unexpected finding. Cardiac fibrosis is not caused directly by the pathogen itself but by T_{H}2-biased (Avraham et al., 2013; Coutinho et al., 2007; Pesce et al., 2009) and T_{H}17-biased (Barron and Wynn, 2011; Lei et al., 2016; Ray et al., 2014; Wang et al., 2015) immune responses mounted against T. cruzi over time. Vaccination of mice with individually-loaded vector and protein vaccines appeared to accelerate the development of the T_{H}2 and T_{H}17-biased responses that are well-known mediators of fibrosis. In contrast, vaccination of mice with vector+protein-loaded DC, which has previously been shown (Konduri et al., 2016; Decker et al., 2006; Decker et al., 2009) to promote anti-fibrotic T_{H}1-biased (Gowdy et al., 2012; Tao et al., 2009; Zhang et al., 2001) responses, did not generate significant fibrotic damage. Given that there was no vaccine-mediated acceleration of pathogen-specific immune responses among unvaccinated mice, fibrotic damage in this group was also relatively low. Presumably, unvaccinated mice would have eventually developed similar levels of cardiac fibrosis as the vector-only and protein-only loaded groups if sacrificed at a significantly later time point. This result provides a cautionary tale for others attempting amelioration of Chagas by immunotherapeutic means. T_{H}1 polarized adaptive responses may be important, not solely for control of pathogen, but also for minimization of immune mediated damage to vital cardiac tissue.

The acute T. cruzi H1 infection mouse model used for these studies is well established and has been shown by several investigators to induce detectable parasitemia, cardiac parasites, and cardiac inflammation (Limon-Flores et al., 2010; Dumonteil et al., 2004; Martinez-Campos et al., 2015; Barry et al., 2016). While it was found that the dendritic cell vaccine construct did not result in appreciable reductions in blood parasite burdens, a significant reduction in cardiac inflammation was observed, which would translate to improved cardiac health. Parasite burden following immunosuppression would be an important parameter to evaluate if there was evidence that the treatment reduced blood parasite burdens to undetectable levels at key time points, for example at the expected peak parasitemia time (Bustamante et al., 2014; Cencig et al., 2012). However, in this model the blood parasite burden was not reduced to undetectable levels, and thus there was not strong evidence that sterilizing immunity had been achieved.

Further, the vaccine construct used in this model is consistent with DNA and protein based vaccines using the Tc24 and TSA1 antigens that achieve immune control of the parasites and significantly reduce parasite burdens and cardiac pathology but without achieving sterilizing immunity (Dumonteil et al., 2004; Sanchez-Burgos et al., 2007; Martinez-Campos et al., 2015; Barry et al., 2016). As 60-70% of chronically-infected individuals naturally remain in the indeterminate stage without ever developing CCC, many hypothesize that immune control of the parasite through vaccination or other immune modulation, without complete elimination of the parasite, remains a viable option for reducing disease burden and improving cardiac health (Dumonteil et al., 2012; Lee et al., 2012). Despite the fact that sterilizing immunity was not likely achieved with the dendritic cell vaccine constructs described here, these results clearly demonstrate that the generation of antigen specific immune responses against T. cruzi infection through genetic adjuvantation can successfully mitigate cardiac pathology associated with Chagasic cardiomyopathy.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Adra et al., Gene, 60:65-74, 1987.
Armentano et al., Proc. Natl. Acad. Sci. USA, 87(16):6141-6145, 1990.
Avraham et al., FASEB J., 27:1114-1126, 2013.
Barron and Wynn, Am J Physiol Gastrointest Liver Physiol., 300:G723-728, 2011.
Batra et al., Am. J. Respir. Cell Mol. Biol., 21(2):238-245, 1999.
Bett et al., J. Virololgy, 67(10):5911-5921, 1993.
Blackwell et al., Arch. Otolaryngol. Head. Neck Surg., 125(8):856-863, 1999.
Bryan et al., PLoS Negl Trop Dis., 4:e733, 2010.
Bustamante et al., J Infect Dis., 209:150-162, 2014.
Caldas et al., Acta Trop., 123:170-177, 2012.
Cencig et al., PLoS Negl Trop Dis., 5:e1216, 2011.
Cencig et al., Int J Antimicrob Agents, 40:527-532, 2012.
Chillon et al., J Virol., 73(3):2537-2540, 1999.
Choi et al., Stem Cells 19, No. 3, 236-246, 2001.
Coutinho et al., J Infect Dis., 195:288-295, 2007.
de Felipe et al., Traffic, 5: 616-626, 2004.
Decker et al., Vaccine, 24:3203-3216, 2006.
Decker et al., Blood, 113:4213-4223, 2009.
Dobson et al., Nucleic Acids Res. 10:2635-2637, 1982.
Dorai et al., Int. J. Cancer, 82(6):846-852, 1999.
Dumonteil et al., Infect Immun., 72:46-53, 2004.
Dumonteil et al., Expert Rev Vaccines, 11:1043-1055, 2012.
el Bouhdidi et al., Parasite Immunol., 16:69-76, 1994.
Farrow et al., PLoS Negl Trop Dis., 8:e3089, 2014.
Feldman et al., Semin. Interv. Cardiol., 1(3):203-208,1996.
Fujiwara and Tanaka, Nippon Geka Gakkai Zasshi, 99(7):463-468, 1998.
Gangisetty and Reddy, J Neurosci Methods, 181:58-66, 2009.
Garzoni et al., J Infect Dis., 197:906-915, 2008.
Gowdy et al., Am J Respir Cell Mol Biol., 46:249-256, 2012.
Graham and Prevec, Mol. Biotechnol., 3(3):207-220, 1995.
Gunning et al., Proc. Natl. Acad. Sci. USA, 84:4831-4835, 1989.
Gupta and Garg. PLoS One, 8:e59434, 2013.
Haecker et al., Hum. Gene Ther., 7(15):1907-1914, 1996.
Han et al., Euro. J. Surgical Oncology, 25:194-198, 1999.
Hanks et al., Nat Med., 11:130-137, 2005.
Hawley, et al., Gene Ther., 1:136-138, 1994.
Hawley et al., Proc. Natl. Acad. Sci. USA, 93:10297-10302, 1996.
Hermens and Verhaagen, Prog. Neurobiol., 55(4):399-432, 1998.
Irie et al., Antisense Nucleic Acid Drug Dev., 9(4):341-349, 1999.
Stewart et al., Gene Ther., 6(3):350-363, 1999.
Karasuyama et al., J. Exp. Med., 169:13, 1989.
Keller et al., Blood, 92:877-887, 1998.
Kemnade et al., Mol Ther., 20:1462-1471, 2012.
Kim et al., PLoS One, 6:e18556, 2011.
Konduri et al., J Infect Dis., 207:1764-1772, 2013.
Konduri et al., Oncoimmunology, 5:e1213933, 2016.
Krisky et al., Gene Ther., 5: 1517-30, 1998.
Lee et al., Hum Vaccin Immunother., 8:1293-1301, 2012.
Lei et al., Clin Exp Rheumatol., 34 Suppl. 100:14-22, 2016.
Lesch, Biol. Psychiatry, 45(3):247-253, 1999.
Limon-Flores et al., Vaccine, 28:7414-7419, 2010.
Lin et al., Nat Med., 11:387-393, 2005.
Marienfeld et al., Gene Ther., 6(6):1101-1113, 1999.
Martin and Tarleton, Immunol Rev., 201:304-317, 2004.
Martinez-Campos et al., Vaccine, 33:4505-4512, 2015.
Melo et al., Parasit Vectors, 8:154, 2015.
Morrison et al., J. Gen. Virol., 78(Pt 4):873-878, 1997.
Narayanan et al., J Clin Invest. 121:1524-1534, 2011.
Neilson, Nat Med., 11:373-374, 2005.
Padilla et al., Curr Opin Immunol., 21:385-390, 2009.
Parks et al., J. Virol., 71(4):3293-8, 1997.
Pesce et al., PLoS Pathog., 5:e1000371, 2009.
Petrof, Eur. Respir. J., 11(2):492-497, 1998.
Pfeifer, A. and I. M. Verma, Annu. Rev. Genomics Hum. Genet., 2:177-211, 2001.
Piron et al., Acta Trop., 103:195-200, 2007.
Quijano-Hernandez et al., Ann N Y Acad Sci., 1149:343-346, 2008.
Ramachandran et al., J Immunol., 186:3934-3945, 2011.
Ray et al., Curr Opin Gastroenterol., 30:531-538, 2014.
Reddy et al., J. Virol., 72(2):1394-1402, 1998.
Robbins and Ghivizzani, Pharmacol. Ther., 80(1):35-47, 1998.
Robbins et al., Proc. Natl. Acad. Sci. USA, 95(17):10182-10187 1998.
Robbins et al., Trends Biotechnol., 16(1):35-40, 1998.
Shi and Massague, Cell, 113:685-700, 2003.
Singer-Sam et al., 1984. Gene 32:409-417
Smith, Arch. Neurol., 55(8):1061-1064, 1998.
Sun et al., Mol Ther., 18:2006-2017, 2010.
Tao et al., Vaccine, 27:4110-4117, 2009.
Udoko et al., PLoS Negl Trop Dis., 10:e0003747, 2016.
U.S. Pat. No. 4,937,190
U.S. Pat. No. 5,670,488
U.S. Pat. No. 5,824,544
U.S. Pat. No. 5,932,210
U.S. Pat. No. 6,218,181
Vanderkwaak et al., Gynecol. Oncol., 74(2):227-234, 1999.
Waghabi et al., Cell Microbiol., 7:511-517, 2005.
Wang et al., PLoS Negl Trop Dis., 9:e0003434, 2015.
Wilson, J Clin. Invest., 98(11):2435, 1996.
Wu, Chung Hua Min Kuo Hsiao Erh Ko I Hsueh Hui Tsa Chih, 39(5):297-300, 1998.
Zhang et al., Semin Immunol., 12:361-378, 2000.
Zhang et al., Gene Ther., 8:1333-1342, 2001.
Zheng et al., J. Gen. Virol., 80(Pt 7):1735-1742, 1999.
Zhou and Tedder, Blood, 3295-3301, 1995.
Zhou and Tedder, Journal of Immunology, 3821-3835, 1995.

What is claimed is:

1. An antigen presenting cell comprising the recombinant nucleic acid encoding an expression vector for expression of least a first antigen from an intracellular pathogen.

2. The cell of claim 1, wherein the cell is a dendritic cell.

3. The cell of claim 1, wherein the cell is a human cell.

4. The cell of claim 3, wherein the cell is a human dendritic cell.

5. The cell of claim 1, wherein the antigen presenting cell further comprises a recombinant antigen from the intracellular pathogen.

6. The cell of claim 5, wherein the antigen presenting cell comprises said expression vector and further comprises a recombinant protein including at least a portion of said first antigen from the intracellular pathogen.

7. The cell of claim 6, wherein the first antigen is from an intracellular bacteria.

8. The cell of claim 7, wherein the a first antigen is an antigen from Bartonella henselae, Francisella tularensis, Listeria monocytogenes, Salmonella Typhi, Brucella, Legionella, a Mycobacterium, Nocardia, Rhodococcus equi, Yersinia, Chlamydia, Rickettsia or Coxiella.

9. The cell of claim 8, wherein the a first antigen is an antigen from Mycobacterium leprae or Mycobacterium tuberculosis.

10. The cell of claim 1, wherein the first antigen is from a virus.

11. The cell of claim 10, wherein the first antigen is from a herpes virus or a retrovirus.

12. The cell of claim 11, wherein the first antigen is from HIV.

13. The cell of claim 1, wherein the first antigen is from a fungus.

14. The cell of claim 13, wherein the first antigen is from Histoplasma capsulatum, Cryptococcus neoformans or Pneumocystis jirovecii.

15. The cell of claim 1, wherein the first antigen is from a protozoa.

16. The cell of claim 1, wherein the first antigen is from a Apicomplexan or a Trypanosomatid.

17. The cell of claim 16, wherein the first antigen is from a Plasmodium spp., Toxoplasma gondii, Cryptosporidium parvum or Leishmania spp.

18. The cell of claim 16, wherein the first antigen is a T. cruzi antigen.

19. The cell of claim 16, wherein the first antigen is SA85-L1, Tc52, TSA1 or Tc24.

20. The cell of claim 19, wherein the first antigen is a T. cruzi Tc24 antigen.

21. The cell of any one of claims 1-20, wherein the cell comprises an expression vector of at least a second antigen from the pathogen

22. The cell of claim 21, wherein the first antigen and the second antigen are encoded by the same expression vector.

23. The cell of claim 21, wherein the first antigen and the second antigen are encoded as a fusion protein.

24. The cell of any one of claims 1-20, wherein the cell comprises an expression vector that encodes a genetic adjuvant.

25. The cell of claim 24, wherein the genetic adjuvant comprises the coding sequence for constitutively activated AKT (caAKT), inducible MyD88/CD40 (iMC), or dominant-negative SHP-1 (dnSHP).

26. The cell of claim 25, wherein the genetic adjuvant is dnSHP.

27. The cell of claim 24, wherein the genetic adjuvant is encoded on the same vector as the first antigen.

28. The cell of claim 27, wherein the genetic adjuvant is upstream of the sequence encoding the antigen.

29. An immunogenic composition comprising the antigen-presenting cells of any of claims 1-28 and a pharmaceutically acceptable carrier.

30. A method for treating or preventing an infection with an intracellular pathogen in a subject comprising administering to the subject an effective amount of antigen presenting cells of any of claims 1-28.

31. A recombinant nucleic acid comprising a sequence encoding at least a first antigen from T. cruzi and a sequence encoding a genetic adjuvant.

32. The recombinant nucleic acid of claim 31, wherein the antigen is selected from SA85-L1, Tc52, TSA1 or Tc24.

33. The recombinant nucleic acid of claim 31, wherein the nucleic acid sequence encodes at least two antigens from T. cruzi.

34. The recombinant nucleic acid of claim 33, wherein the at least two antigens are encoded as a fusion protein.

35. The recombinant nucleic acid of claim 33, wherein the at least two antigens comprise Tc24 and TSA1 antigens of T. cruzi.

36. The recombinant nucleic acid of claim 31, wherein the genetic adjuvant comprises the coding sequence for constitutively activated AKT (caAKT), inducible MyD88/CD40 (iMC), or dominant-negative SHP-1 (dnSHP).

37. The recombinant nucleic acid of claim 36, wherein the genetic adjuvant is dnSHP.

38. The recombinant nucleic acid of claim 31, wherein the sequence encoding a genetic adjuvant is upstream of the sequence encoding the antigen.

39. An expression vector comprising the recombinant nucleic acid of any of claims 31-38.

40. The expression vector of claim 39, wherein the expression vector is an adenoviral, adeno associated, or retroviral vector.

41. The expression vector of claim 40, wherein the expression vector is an adenoviral vector.

42. A host cell comprising the recombinant nucleic acid of any of claims 31-38 or the expression vector of claims 39-41.

43. The host cell of claim 42, wherein the host cell is an antigen presenting cell.

44. The host cell of claim 43, wherein the cell is a dendritic cell.

45. The host cell of claim 43, wherein the cell is a human cell.

46. The host cell of claim 44, wherein the cell is a human dendritic cell.

47. The host cell of claim 43, wherein the antigen presenting cell further comprises a recombinant T. cruzi antigen.

48. The host cell of claim 47, wherein the recombinant T. cruzi antigen comprises Tc24 protein.

49. A composition comprising antigen-presenting cells, wherein the antigen-presenting cells comprise at least a first T. cruzi polypeptide antigen and a genetic adjuvant.

50. The composition of claim 49, wherein the antigen is selected from. SA85-L1, Tc52, TSA1 or Tc24.

51. The composition of claim 49, wherein the cells comprise at least two antigens from T. cruzi.

52. The composition of claim 51, wherein the at least two antigens are a fusion protein.

53. The composition of claim 51, wherein the at least two antigens comprise Tc24 and TSA1 antigens of T. cruzi.

54. The composition of claim 53, wherein the Tc24 and TSA1 genes are present on an expression vector in said antigen-presenting cells.

55. The composition of claim 49, wherein the cells are dendritic cells.

56. The composition of claim 54, wherein the cells are human cells.

57. The composition of claim 55, wherein the cells are human dendritic cells.

58. The composition of claim 54 wherein the expression vector is an adenoviral vector.

59. The composition of claim 49, wherein the genetic adjuvant is constitutively activated AKT (caAKT), inducible MyD88/CD40 (iMC), or dominant-negative SHP-1 (dnSHP).

60. The composition of claim 59, wherein the genetic adjuvant is dnSHP.

61. The composition of claim 60, wherein the dnSHP is kinase-inactivated SHP1.

62. The composition of claim 54, wherein the gene encoding the genetic adjuvant is present in the same expression vector as the genes encoding Tc24 and TSA1.

63. The composition of claim 62, wherein the gene encoding the genetic adjuvant is upstream of the genes encoding Tc24 and TSA1.

64. The composition of claim 49, wherein the antigen-presenting cells are mature dendritic cells.

65. The composition of claim 49, wherein the antigen presenting cells have been loaded with exogenous recombinant Tc24 protein.

66. An immunogenic composition comprising the antigen-presenting cells of any of claims 49-65 and a pharmaceutically acceptable carrier.

67. A method for treating or preventing an infection with an intracellular pathogen in a subject comprising administering to the subject an effective amount of antigen presenting cells, said cells comprising comprise at least a first polypeptide antigen from the intracellular pathogen and an expression vector encoding said first polypeptide antigen.

68. A method for treating or preventing Chagas disease comprising administering to a subject that has, may have, or is likely to contract Chagas disease a therapeutically effective amount of the composition of claim 66.

69. A method for treating or preventing Chagasic cardiomyopathy comprising administering to a patient with Chagas disease a therapeutically effective amount of the composition of claim 66.

70. A method for reducing T. cruzi parasite burden in a subject infected with T. cruzi comprising administering to the subject a therapeutically effective amount of the composition of claim 66.

71. The method of any of claims 67-70, wherein the method further comprises administering at least a second treatment for T. cruzi infection to the subject.

72. The method of claim 71, wherein the second treatment is a trypanocidal, chemotherapeutic, or immunotherapeutic treatment.

73. The method of claim 72, wherein the trypanocidal treatment is treatment with benznidazole or nifurtimox.

74. The method of claim 29, wherein the chemotherapeutic treatment is treatment with posaconazole.

75. The method of claim 72, wherein the immunotherapeutic treatment is a second administration of the vaccine of claim 66.

76. The method of claim 72, wherein the immunotherapeutic treatment is vaccination with the SA85-L1, Tc52, TSA1, or Tc24 antigens.

77. A method for inducing an immune response in a subject comprising administering to the subject a therapeutically effective amount of the composition of claim 66.

78. A method for preparing T. cruzi antigen presenting dendritic cells comprising:
(a) obtaining immature antigen presenting cell precursors from a subject;
(b) culturing the immature antigen presenting cell precursors to induce differentiation into immature dendritic cells;
(c) transducing the immature dendritic cells with a vector for the expression of T. cruzi antigens;
(d) contacting the immature dendritic cells with a recombinant T. cruzi antigen; and
(e) culturing the immature dendritic cells to produce mature dendritic cells.

79. The method of claim 78, wherein the T. cruzi antigens encoded by the vector are Tc24 and TSA1.

80. The method of claim 78, wherein the recombinant T. cruzi antigen is recombinant Tc24.

81. The method of claim 78, wherein the cells are transduced with the vector and contacted with recombinant T. cruzi antigen together.

82. The method of claim 78, wherein the cells are transduced with the vector prior to contact with the recombinant T. cruzi antigen.

83. The method of claim 78, wherein the cells are contacted with the recombinant T. cruzi antigen prior to transduction with the vector.

84. The method of claim 78, wherein the vector further comprises a gene encoding a genetic adjuvant.

85. The method of claim 84, wherein the genetic adjuvant is constitutively activated AKT (caAKT), inducible MyD88/CD40 (iMC), or dominant-negative SHP-1 (dnSHP).

86. The method of claim 85, wherein the genetic adjuvant is dnSHP.

87. The method of claim 86, wherein the dnSHP is kinase inactivated dominant negative SHP-1.

88. The method of claim 78, wherein the vector is a viral vector.

89. The method of claim 88, wherein the vector is an adenoviral, adeno associated, or retroviral vector.

90. The method of claim 89, wherein the vector is an adenoviral vector.

91. A composition comprising the mature dendritic cells produced by the method of any of claims 78-90 and a pharmaceutically acceptable carrier.

92. A method for treating or preventing Chagas disease comprising administering to a subject that has, may have, or is likely to contract Chagas disease a therapeutically effective amount of the composition of claim 91.

93. A method for treating or preventing Chagasic cardiomyopathy comprising administering to a subject with Chagas disease a therapeutically effective amount of the composition of claim 91.

94. A method for reducing T. cruzi parasite burden in a subject infected with T. cruzi comprising administering to the subject a therapeutically effective amount of the composition of claim 91.

95. The method of any of claims 92-94, wherein the method further comprises administering at least a second treatment for T. cruzi infection to the subject.

96. The method of claim 95, wherein the second treatment is a trypanocidal, chemotherapeutic, or immunotherapeutic treatment.

97. The method of claim 96, wherein the trypanocidal treatment is treatment with benznidazole or nifurtimox.

98. The method of claim 51, wherein the chemotherapeutic treatment is treatment with posaconazole.

99. The method of claim 96, wherein the immunotherapeutic treatment is a second administration of the vaccine of claim 91.

100. The method of claim 96, wherein the immunotherapeutic treatment is vaccination with SA85-L1, Tc52, TSA1, or Tc24 antigens.

101. A method for inducing an immune response in a subject comprising administering to the subject a therapeutically effective amount of the vaccine of claim 91.
